(19) United States
(12) Patent Application Publication
(10) Pub. No.: US 2022/0370107 A1
(43) Pub. Date: Nov. 24, 2022

(54) ADVANCED TOOLS FOR BONE CONDUCTION IMPLANTATION

(71) Applicant: COCHLEAR LIMITED, Macquarie University, NSW (AU)

(72) Inventors: Stina MILLGÅRD, Macquarie University, NSW (AU); Stefan MAGNANDER, Macquarie University, NSW (AU)

(21) Appl. No.: 17/771,617

(22) PCT Filed: Oct. 23, 2020
(86) PCT No.: PCT/IB2020/059984

§ 371 Date: Apr. 25, 2022

Related U.S. Application Data
(60) Provisional application No. 62/926,050, filed on Oct. 25, 2019.

(51) Int. Cl. A61B 17/86

(52) U.S. Cl. CPC A61B 17/86; H04R 2460/13

(57) ABSTRACT

A device, including a body including a torque transfer section and a torque receiver section, wherein the device is a bone conduction hearing prosthesis bone fixture implant insertion device configured to interface with an interior of the bone fixture, and the device is made of a same class of materials at the torque transfer section and the torque receiver section and section(s) that interface with the bone fixture.

### CROSS-REFERENCE TO RELATED APPLICATIONS

This application claims priority to U.S. Provisional Application No. 62/926,050, entitled ADVANCED TOOLS FOR BONE CONDUCTION IMPLANTATION, filed on Oct. 25, 2019, naming Stina MILLGÅRD of Mölnlycke, Sweden as an inventor, the entire contents of that application being incorporated herein by reference in its entirety.

### BACKGROUND

### Field of the Invention

Some embodiments relate generally to prostheses and, more particularly, to a prosthesis having a bone fixture and/or an abutment.

### Related Art

For persons who cannot benefit from traditional acoustic hearing aids, there are other types of commercially available hearing prostheses such as, for example, bone conduction hearing prostheses (commonly referred to as “bone conduction devices”). Bone conduction devices mechanically transmit sound information to a recipient's cochlea by transferring vibrations to a person's skull. This enables the hearing prosthesis to be effective regardless of whether there is disease or damage in the middle ear.

Traditionally, bone conduction devices transfer vibrations from an external vibrator to the skull through a bone conduction implant that penetrates the skin and is physically attached to both the vibrator and the skull. Typically, the external vibrator is connected to the percutaneous bone conduction implant located behind the outer ear facilitating the efficient transfer of sound via the skull to the cochlea. The bone conduction implant connecting the vibrator to the skull generally comprises two components: a bone attachment piece (e.g., bone fixture/fixture) that is attached or implanted directly to the skull, and a skin penetrating piece attached to the bone attachment piece, commonly referred to as an abutment.

### SUMMARY

In an exemplary embodiment, there is a device, comprising a body including a torque transfer section and torque receiver section, wherein the device is a bone conduction hearing prosthesis bone fixture implant insertion device configured to interface with an interior of the bone fixture and the device is made of a same class of materials at the torque transfer section and the torque receiver section and section(s) that interface with the bone fixture.

In an exemplary embodiment, there is a device, comprising a torque transfer section and a torque receiver section, wherein the device is a bone conduction hearing prosthesis abutment implantation device configured to interface with an interior of the abutment, and at least one of: (i) the device is made of like materials at the torque transfer section and the torque receiver section and section(s) that interface with the bone fixture or (ii) the device is configured to retain the abutment to the device and the device is configured such that torque transferred from the device is applied through a component that retains the device to the abutment.

In an exemplary embodiment, there is a method, comprising obtaining a component of an implantable portion of a bone conduction device, supporting the component with a tool via a friction fit and/or interference fit; and attaching the component to a mammal by transferring and/or reacting a torque with the tool.

In an exemplary embodiment, there is a method, comprising obtaining a component of an implantable portion of a bone conduction device, retaining the component to a tool without limb flexing and moving the component to interface with a mammal by moving the tool with the component retained to the tool.

### BRIEF DESCRIPTION OF THE DRAWINGS

Embodiments of the present invention are described herein with reference to the attached drawing sheets in which:

FIG. 1 is a perspective view of a percutaneous bone conduction device in which embodiments of the present invention may be implemented;

FIGS. 2A and 2B and 18 depict some details of the bone conduction device of FIG. 1;

FIG. 3 depicts an exemplary passive transcutaneous bone conduction device;

FIG. 4 depicts an exemplary active transcutaneous bone conduction device;

FIG. 5 depicts a side view of an exemplary bone screw;

FIG. 5A depicts a cross-sectional view of the exemplary bone screw of FIG. 5A;

FIG. 5B depicts an isometric view of a tool in proximity to the bone fixture;

FIGS. 6 and 7 depict views of some exemplary tools;

FIG. 8 depicts a conceptual interface of the tool with a bone fixture;

FIG. 9 depicts a silhouette of a portion of the tool for purposes of discussion;

FIGS. 10-16 depict cross-sectional views of a portion of the tool;

FIG. 17 depicts a non-embodiment for purposes of differentiation;

FIGS. 19-21A depict exemplary embodiments of another tool;

FIG. 22 depicts a tool in proximity with an abutment;

FIGS. 23 and 24 depict exemplary tools interfacing with an abutment;

FIGS. 25 to 30 and 35-36 depict some views of another tool;

FIGS. 31-33 and 35 depict exemplary flowcharts for exemplary methods; and

FIG. 34 depicts a schematic representing deformation.

### DETAILED DESCRIPTION

The teachings detailed herein are implemented in conjunction with sensory prostheses, such as hearing implants specifically, and neural stimulation devices, at least in some instances. Other types of sensory prostheses can include retinal implants. Accordingly, any teaching herein with respect to a sensory prosthesis corresponds to a disclosure of utilizing those teachings in/with a hearing implant and in/with a retinal implant, unless otherwise specified, providing the art enables such. By way of example, the bone fixtures herein can be used to support a processor housing or the like/secure such to bone, which processor (or more generally circuitry—any disclosure herein of a processor corresponds to a disclosure of circuitry (which may not have processing capability)) can be a retinal implant processor. Further, the housing can contain a processor for a pacemaker or another implant, such as a device that releases drugs or other therapeutic substances. Also, the fixture and/or abutment can be part of an artificial limb or a joint or bone reconstruction, or can be a fixture or abutment for a dental implant. Moreover, with respect to any teachings herein, such corresponds to a disclosure of utilizing those teachings with a cochlear implant (e.g., the fixture can support an housing for a processor for such, or for the receiver stimulator, or for a microphone (implanted), or for the inductance coil, or for some other antenna, etc.), a bone conduction device (active and/or passive transcutaneous bone conduction devices, and/or percutaneous bone conduction devices) and a middle ear implant (the fixture can support an actuator, or a housing for a processor, or a stimulator unit, or the inductance coil/antenna thereof, or for a microphone (implanted) etc.), or a sleep-apnea device, such as an implanted housing for such a device, which may include circuitry (and disclosure of a processor herein corresponds to an alternate embodiment of circuitry for the given prosthesis) providing that the art enables such, unless otherwise noted. To be clear, any teaching herein with respect to a specific sensory prosthesis corresponds to a disclosure of utilizing those teachings in/with any of the aforementioned hearing prostheses, and/or the other prosthesis just detailed, and vice versa. Corollary to this is at least some teachings detailed herein can be implemented in somatosensory implants and/or chemosensory implants. Accordingly, any teaching herein with respect to a sensory prosthesis corresponds to a disclosure of utilizing those teachings with/in a somatosensory implant and/or a chemosensory implant and/or the other prostheses detailed above.

While the teachings detailed herein will be described for the most part with respect to hearing prostheses, in keeping with the above, it is noted that any disclosure herein with respect to a hearing prosthesis corresponds to a disclosure of another embodiment of utilizing the associated teachings with respect to any of the other prostheses noted herein, whether a species of a hearing prosthesis, or a species of a sensory prosthesis, such as a retinal prosthesis. In this regard, any disclosure herein with respect to a device for evoking a hearing percept corresponds to a disclosure of a device for evoking other types of neural percepts in other embodiments, such as a visual/sight percept, a tactile percept, a smell precept or a taste percept, unless otherwise indicated and/or unless the art does not enable such. Any disclosure herein of a device, system, and/or method that is used to or results in ultimate stimulation of the auditory nerve corresponds to a disclosure of an analogous stimulation of the optic nerve utilizing analogous components/methods/systems.

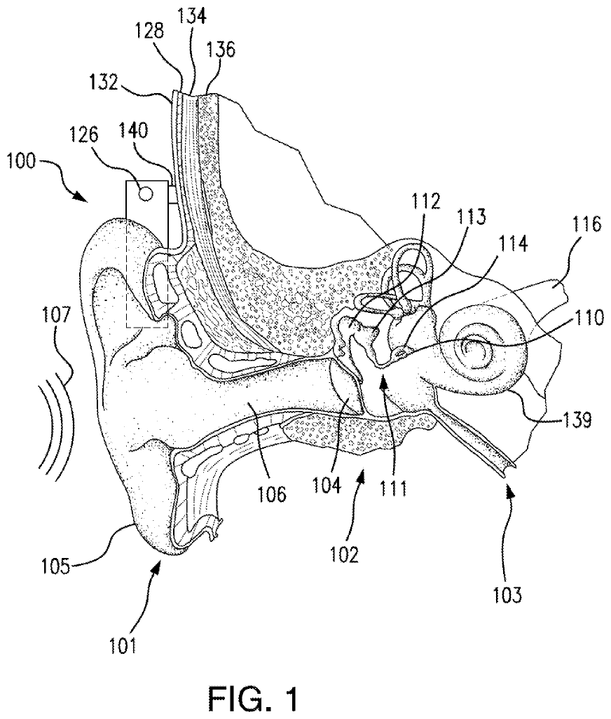

FIG. 1 is a perspective view of a bone conduction device 100 in which embodiments of the present invention can be implemented. As shown, the recipient has an outer ear 101, a middle ear 102 and an inner ear 103. Elements of outer ear 101, middle ear 102, and inner ear 103 are described below, followed by a description of bone conduction device 100.

In a fully functional human hearing anatomy, outer ear 101 comprises an auricle 105 and an ear canal 106. A sound wave or acoustic pressure 107 is collected by auricle 105 and channeled into and through ear canal 106. Disposed across the distal end of ear canal 106 is a tympanic membrane 104 which vibrates in response to acoustic wave 107. This vibration is coupled to oval window or fenestra ovalis 210 through three bones of middle ear 102, collectively referred to as the ossicles 111 and comprising the malleus 112, the incus 113 and the stapes 114. The ossicles 111 of middle ear 102 serve to filter and amplify acoustic wave 107, causing oval window 210 to vibrate. Such vibration sets up waves of fluid motion within cochlea 139. Such fluid motion, in turn, activates hair cells (not shown) that line the inside of cochlea 139. Activation of the hair cells causes appropriate nerve impulses to be transferred through the spiral ganglion cells and auditory nerve 116 to the brain (not shown), where they are perceived as sound.

FIG. 1 also illustrates the positioning of bone conduction device 100 relative to outer ear 101, middle ear 102 and inner ear 103 of a recipient of device 100. As shown, bone conduction device 100 is positioned behind outer ear 101 of the recipient and comprises a sound input element 126 to receive sound signals. Sound input element can comprise, for example, a microphone, telecoil, etc. In an exemplary embodiment, sound input element 126 can be located, for example, on or in bone conduction device 100, or on a cable extending from bone conduction device 100.

In an exemplary embodiment, bone conduction device 100 comprises an operationally removable component and a bone conduction implant. The operationally removable component is operationally releasably coupled to the bone conduction implant. By operationally releasably coupled, it is meant that it is releasable in such a manner that the recipient can relatively easily attach and remove the operationally removable component during normal use of the bone conduction device 100. Such releasable coupling is accomplished via a coupling apparatus of the operationally removable component and a corresponding mating apparatus of the bone conduction implant, as will be detailed below. This as contrasted with how the bone conduction implant is attached to the skull, as will also be detailed below. The operationally removable component includes a sound processor (not shown), a vibrating electromagnetic actuator and/or a vibrating piezoelectric actuator and/or other type of actuator (not shown—which are sometimes referred to herein as a vibrator, corresponding to a genus of which these are species of) and/or various other operational components, such as sound input device 126. In this regard, the operationally removable component is sometimes referred to herein as a vibrator unit. More particularly, sound input device 126 (e.g., a microphone) converts received sound signals into electrical signals. These electrical signals are processed by the sound processor. The sound processor generates control signals which cause the actuator to vibrate. In other words, the actuator converts the electrical signals into mechanical motion to impart vibrations to the recipient's skull. It is noted that in some embodiments, the operationally removable component is a vibration sensor. In this regard, the operationally removable component can be a transducer, which is a genus that includes at least the species vibration sensor and vibrator.

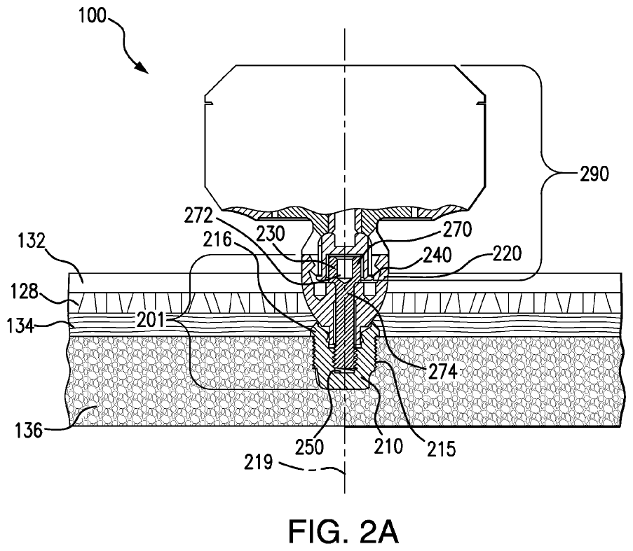

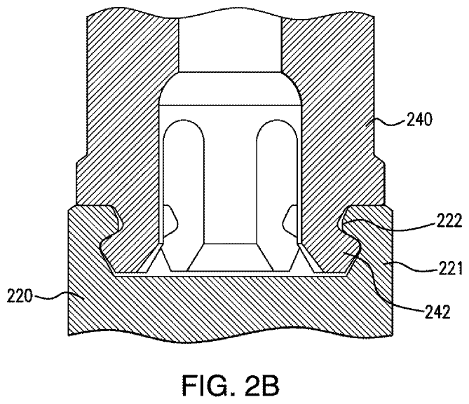

As illustrated, the operationally removable component of the bone conduction device 100 further includes a coupling apparatus 140 configured to operationally removably attach the operationally removable component to a bone conduction implant (also referred to as an anchor system and/or a fixation system) which is implanted in the recipient. In the embodiment of FIG. 1, coupling apparatus 140 is coupled to the bone conduction implant (not shown) implanted in the recipient in a manner that is further detailed below with respect to exemplary embodiments of the bone conduction implant. Briefly, now with reference to FIG. 2A, an exemplary bone conduction implant 201 can include a percutaneous abutment attached to a bone fixture via a screw, the bone fixture being fixed to the recipient's skull bone 136. The abutment extends from the bone fixture which is screwed into bone 136, through muscle 134, fat 128 and skin 132 so that the coupling apparatus can be attached thereto. Such a percutaneous abutment provides an attachment location for the coupling apparatus that facilitates efficient transmission of mechanical force.

As illustrated, the operationally removable component of the bone conduction device 100 further includes a coupling apparatus 140 configured to operationally removably attach the operationally removable component to a bone conduction implant (also referred to as an anchor system and/or a fixation system) which is implanted in the recipient. In the embodiment of FIG. 1, coupling apparatus 140 is coupled to the bone conduction implant (not shown) implanted in the recipient in a manner that is further detailed below with respect to exemplary embodiments of the bone conduction implant. Briefly, now with reference to FIG. 2A, an exemplary bone conduction implant 201 can include a percutaneous abutment attached to a bone fixture via a screw, the bone fixture being fixed to the recipient's skull bone 136. The abutment extends from the bone fixture which is screwed into bone 136, through muscle 134, fat 128 and skin 132 so that the coupling apparatus can be attached thereto. Such a percutaneous abutment provides an attachment location for the coupling apparatus that facilitates efficient transmission of mechanical force.

FIG. 2A depicts additional details of the bone conduction device 100. More particularly, the bone conduction device 100 is shown as including operationally removable component 290 vibrationally connected to and removably coupled to an exemplary bone conduction implant 201 via coupling apparatus 140 (corresponding to coupling apparatus 240) thereof. More particularly, operationally removable component 290 includes a vibrator (not shown) that is in vibrational communication to coupling apparatus 240 such that vibrations generated by the vibrator in response to a sound captured by sound capture device 126 are transmitted to coupling apparatus 240 and then to bone conduction implant 201 in a manner that at least effectively evokes hearing percept. By “effectively evokes a hearing percept,” it is meant that the vibrations are such that a typical human between 18 years old and 40 years old having a fully functioning cochlea receiving such vibrations, where the vibrations communicate speech, would be able to understand the speech communicated by those vibrations in a manner sufficient to carry on a conversation provided that those adult humans are fluent in the language forming the basis of the speech. In an exemplary embodiment, the vibrational communication effectively evokes a hearing percept, if not a functionally utilitarian hearing percept.

Bone conduction implant 201 includes a bone fixture 210 configured to screw into the skull bone 136, a skin-penetrating abutment 220 and an abutment screw 230 that is in the form of an elongate coupling shaft. As may be seen, the abutment screw 230 connects and holds the abutment 220 to the fixture 210, thereby rigidly attaching abutment 220 to bone fixture 210. The rigid attachment is such that the abutment is vibrationally connected to the fixture 210 such that at least some of the vibrational energy transmitted to the abutment is transmitted to the fixture in a sufficient manner to effectively evoke a hearing percept.

It is noted that by way of example only and not by way of limitation, FIG. 2A and the figures thereafter are drawn to scale, although other embodiments can be practiced having different scales.

Some exemplary features of the bone fixture 210 will now be described, followed by exemplary features of the abutment 220 and the abutment screw 230.

Bone fixture 210 (hereinafter sometimes referred to as fixture 210) can be made of any material that has a known ability to integrate into surrounding bone tissue (i.e., it is made of a material that exhibits acceptable osseointegration characteristics). In one embodiment, fixture 210 is formed from a single piece of material and has a main body. In an embodiment, the fixture 210 is made of titanium. The main body of bone fixture 210 includes outer screw threads 215 forming a male screw which is configured to be installed into the skull 136. Fixture 210 also comprises a flange 216 configured to function as a stop when fixture 210 is installed into the skull. Flange 216 prevents the bone fixture 210 in general, and, in particular, screw threads 215, from potentially completely penetrating through the skull. Fixture 210 can further comprise a tool-engaging socket having an internal grip section for easy lifting and handling of fixture 210, as will be described in further detail below. An exemplary tool-engaging socket is described and illustrated in U.S. Provisional Application No. 60/951,163, entitled “Bone Anchor Fixture for a Medical Prosthesis,” filed Jul. 20, 2007, by Applicants Lars Jinton, Erik Holgersson, and Peter Elmberg which, in some embodiments, can be used exactly as detailed therein and/or in a modified form, to install and manipulate the bone fixture 210.

The body of fixture 210 can have a length sufficient to securely anchor the fixture 210 to the skull without penetrating entirely through the skull. The length of the body can therefore depend on the thickness of the skull at the implantation site. In one embodiment, the fixture 210 has a length that is no greater than 5 mm, measured from the planar bottom surface of the flange 216 to the end of the distal region (the portion closest to the brain), which limits and/or prevents the possibility that the fixture 210 might go completely through the skull). In another embodiment, this length can be anywhere from about 3.0 mm to about 5.0 mm.

The distal region of fixture 210 can also be fitted with self-tapping cutting edges (e.g., three edges) formed into the exterior surface of the fixture 210. Further details of the self-tapping features are described in International Patent Application Publication WO 02/09622, and can be used with some embodiments of bone fixtures exactly as detailed therein and/or in a modified form, to configure the fixtures detailed herein to be installed into a skull.

As illustrated in FIG. 2A, flange 216 has a planar bottom surface for resting against the outer bone surface, when bone fixture 210 has been screwed down into the skull. Flange 216 can have a diameter which exceeds the peak diameter (maximum diameter) of the screw threads 215 (the screw threads 215 of the fixture 210 can have a maximum diameter of about 3.5 to about 5.0 mm). In one embodiment, the diameter of the flange 216 exceeds the peak diameter of the screw threads 215 by approximately 10-20%. Although flange 216 is illustrated in FIG. 2A as being circular, flange 216 can be configured in a variety of shapes so long as flange 216 has a diameter or width that is greater than the peak diameter of the screw threads 215. Also, the size of flange 216 can vary depending on the particular application for which the bone conduction implant 201 is intended.

As may be seen in FIG. 2A, the outer peripheral surface of flange 216 has a cylindrical part and a flared top portion. The upper end of flange 216 is designed with an open cavity having a tapered inner side wall. The tapered inner side wall 217 is adjacent to the grip section (not shown). The interior of the fixture 210 further includes an inner lower bore 250 having female screw threads for securing a coupling shaft of abutment screw 230 (described further below). As may be seen, the fixture 210 further includes an inner upper bore 260 that receives a bottom portion of abutment 220.

In an exemplary embodiment, the flange 216 can be in the form of a protruding hex instead of being circular. That is, flange 216 can have a hexagonal cross-section that lies on a plane normal to the longitudinal axis 219 of the bone fixture 220/bone conduction implant 201 such that a female hex-head socket wrench can be used to apply torque to the bone fixture 210. However, in the embodiment illustrated in FIG. 2A, the flange 216 has a smooth, upper end that has a circular cross-section that lies on the aforementioned plane, and thus does not have a protruding hex. The smooth upper end of the flange 216 and the absence of any sharp corners provides for improved soft tissue adaptation. As mentioned above, flange 216 also comprises a cylindrical part which, together with the flared upper part, provides sufficient height in the longitudinal direction for connection with the abutment 220.

It is noted that the bone fixture depicted in FIG. 2A and the following figures are exemplary. Any bone fixture of any type, size/having any geometry can be used in some embodiments providing that the bone fixture permits embodiments as detailed herein and variations thereof to be practiced.

As noted above, bone conduction implant 201 further includes an abutment screw 230 as depicted in FIG. 2A. Abutment screw 230 includes a screw head 270 that has an internal upper bore 272 that can form a unigrip, internal hex or multi-lobular configuration for a cooperating insertion tool (not illustrated here). The screw head 270 is connected to elongate member 274 that extends downward as shown. At the bottom of the abutment screw 230 are male screw thread formed in the elongate member 274. These male screw threads are dimensioned to interact with the corresponding female threads of inner lower bore of bone fixture 210. Upon application of a tightening torque to abutment screw 230, screw head 270 reacts against the corresponding surface of abutment 220 to pull abutment 220 to fixture 210, as will be described further below.

In an exemplary embodiment, the screw head 270 includes male screw threads (not shown) thereabout, although other embodiments do not include such screw threads. While the embodiment depicted in FIG. 2A does not utilize those screw threads for removable attachment of the operationally removable component 290 to the bone conduction implant 201 (the coupling apparatus 240 generally does not contact the screw head 270 in some embodiments, and slides along the outside of the threads during installation in other embodiments), in some embodiments, the screw threads have utility in, for example, diagnostic methods and/or therapeutic methods.

It is noted that the abutment screw depicted in FIG. 2A and the following figures are exemplary. Any abutment screw of any type, size/having any geometry can be used in some embodiments providing that the abutment screw permits embodiments as detailed herein and variations thereof to be practiced.

As noted above, bone conduction implant 201 further includes an abutment 220 as depicted in FIG. 2A. In the embodiment of FIG. 2A, abutment 220 is symmetrical with respect to at least those portions above the top portion of the bone fixture 210. In this regard, the exterior surfaces of abutment 220 depicted in FIG. 2A form concentric outer profiles about longitudinal axis 219. As may be seen, the exterior surfaces of abutment 220 establish diameters lying on planes normal to longitudinal axis 219 that vary along the length of longitudinal axis 219. More specifically, abutment 220 includes outer diameters that progressively become larger with increased height until about the portions proximate the end. In other embodiments, the outer diameters become progressively larger until the end, and other embodiments can have other outer profiles. In an exemplary embodiment, the abutment can correspond to any of those detailed in U.S. patent application Ser. No. 13/270,691, filed Oct. 11, 2011, by Applicants Goran Bjorn, Stefan Magnander, and Dr. Marcus Andersson, and/or variations thereof. Any abutment of any configuration can be utilized in some embodiments providing that those embodiments enable the teachings detailed herein and/or variations thereof to be practiced.

The bottom of the abutment 220 includes a fixture connection section extending below a reference plane extending across the top of fixture 210 that interfaces with fixture 210. Upon sufficient tensioning of abutment screw 230, abutment 220 sufficiently elastically and/or plastically stresses bone fixture 210, and/or vice versa, so as to form an effectively hermetic seal at the interface of surfaces of the abutment 220 and fixture 210. Such can reduce (including eliminate) the chances of micro-leakage of microbes into the gaps between the abutment 220, fixture 210 and abutment screw 230.

As noted above, the bone conduction device 100 is configured such that the operationally removably component 290 is removably attached to the implant 201. This is accomplished via a coupler, a portion of which is included in the bone conduction implant 201, and a portion of which is included in the operationally removable component 290 (e.g., coupling apparatus 240). In an exemplary embodiment, the operationally removable component 290 snap-couples to the abutment 220. FIG. 2B depicts a snap-coupling arrangement utilized with the coupling apparatus 240, although some elements of the bone conduction device 100 are not shown for clarity. More particularly, FIG. 2B depicts a close-up view of the interface between the abutment 220 and the coupling apparatus 240. As may be seen, abutment 220 includes a recess formed by sidewall 221 that has an overhang 222 that interfaces with corresponding teeth 242 of coupling apparatus 240. Teeth 242 elastically deform inward upon the application of sufficient removal and/or installation force to the coupling apparatus 240. In an exemplary embodiment, element 220 can correspond to any abutment herein and variations thereof providing that it includes the snap-coupling arrangement and variations thereof.

It is noted that while the male component is depicted as being a part of the coupling apparatus 240 and the female component is depicted as part of the abutment, in other embodiments, this can be reversed. It is noted that the coupling arrangement of FIGS. 2A and 2B can be used with any of the embodiments of the adapters detailed herein, some examples of such use being detailed below.

In the embodiment of FIGS. 2A and 2B, the connection between the coupling apparatus 240 and the abutment 220 is such that vibrations generated by the operationally removable component 290 (e.g., such as those generated by an electromagnetic actuator and/or a piezoelectric actuator, etc.) in response to a captured sound are effectively communicated to the abutment 220 so as to effectively evoke a hearing percept, if not evoke a functionally utilitarian hearing percept. Such communication can be achieved via a coupling (sometimes referred to herein as a connection) that establishes at least a modicum of rigidity between the two components. In this vein, the dimensions and/or geometries of the interfacing portions are, in at least some embodiments, such that they can be varied in only minor ways while still achieving the utilitarian functionality of the bone conduction device. Put another way, the design of the abutment 220 is such that it will utilitarianly interface with a limited number of designs of coupling apparatus 240. That is, coupling apparatuses of different designs may not utilitarianly couple to the abutment 220, yet there can be utility in coupling removable components having such coupling apparatuses of different designs to the abutment 220. With this in mind, it is noted that there is utilitarian value in not removing the abutment 220 from the recipient, such as, for example, in the case where the abutment is integrated to skin of the recipient.

As may be seen from FIGS. 2A and 2B, the abutment 220 forms a female portion of the coupling of the bone conduction device 100, and the coupling apparatus 240 forms a male portion of the coupling. Some operationally removable components different from component 290 have coupling portions that are female instead of male, and thus are generally incompatible for coupling directly to the abutment 220. An exemplary embodiment provides an adapter that is configured to enable coupling of such an operationally removable component to the abutment 220, as will now be described.

It is noted that the bone fixture depicted in FIGS. 2A and 2B and the following FIGs. are exemplary. Any bone fixture and/or pedestal (an alternate embodiment can be a pedestal, such as in US patent application publication No. 62784081—some exemplary embodiments include tools to interface with such and methods of handling such and implanting such) of any type, size/having any geometry can be used in some embodiments providing that the bone fixture permits embodiments as detailed herein and variations thereof to be practiced.

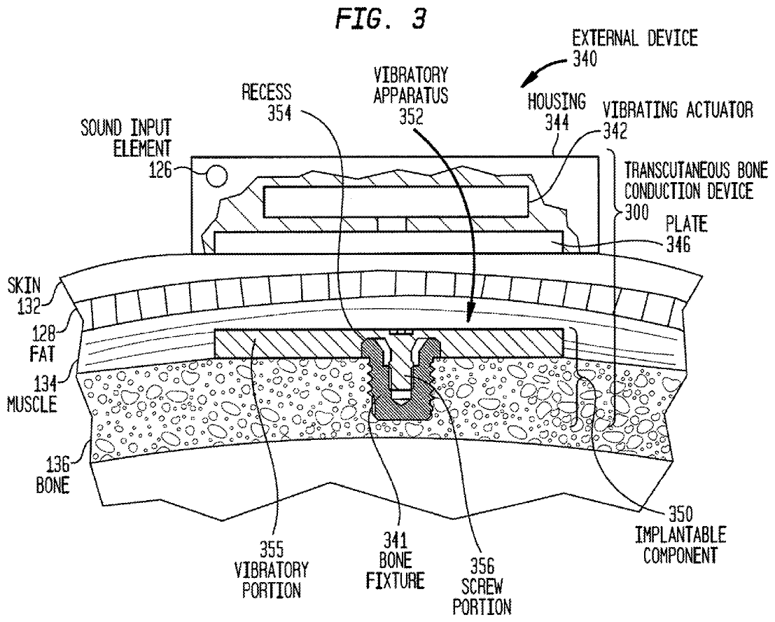

FIG. 3 depicts an exemplary embodiment of a transcutaneous bone conduction device 300 according to an embodiment that includes an external device 340 (corresponding to, for example, element 140B of FIG. 1B) and an implantable component 350 (corresponding to, for example, element 150 of FIG. 1B). The transcutaneous bone conduction device 300 of FIG. 3 is a passive transcutaneous bone conduction device in that a vibrating electromagnetic actuator 342 is located in the external device 340. Vibrating electromagnetic actuator 342 is located in housing 344 of the external component, and is coupled to plate 346. Plate 346 may be in the form of a permanent magnet and/or in another form that generates and/or is reactive to a magnetic field, or otherwise permits the establishment of magnetic attraction between the external device 340 and the implantable component 350 sufficient to hold the external device 340 against the skin of the recipient.

In an exemplary embodiment, the vibrating electromagnetic actuator 342 is a device that converts electrical signals into vibration. In operation, sound input element 126 converts sound into electrical signals. Specifically, the transcutaneous bone conduction device 300 provides these electrical signals to vibrating actuator 342, or to a sound processor (not shown) that processes the electrical signals, and then provides those processed signals to vibrating electromagnetic actuator 342. The vibrating electromagnetic actuator 342 converts the electrical signals (processed or unprocessed) into vibrations. Because vibrating electromagnetic actuator 342 is mechanically coupled to plate 346, the vibrations are transferred from the vibrating actuator 342 to plate 346. Implanted plate assembly 352 is part of the implantable component 350, and is made of a ferromagnetic material that may be in the form of a permanent magnet, that generates and/or is reactive to a magnetic field, or otherwise permits the establishment of a magnetic attraction between the external device 340 and the implantable component 350 sufficient to hold the external device 340 against the skin of the recipient. Accordingly, vibrations produced by the vibrating electromagnetic actuator 342 of the external device 340 are transferred from plate 346 across the skin to plate 355 of plate assembly 352. This can be accomplished as a result of mechanical conduction of the vibrations through the skin, resulting from the external device 340 being in direct contact with the skin and/or from the magnetic field between the two plates. These vibrations are transferred without penetrating the skin with a solid object such as an abutment as detailed herein with respect to a percutaneous bone conduction device.

As may be seen, the implanted plate assembly 352 is substantially rigidly attached to a bone fixture 341 in this embodiment. Plate screw 356 is used to secure plate assembly 352 to bone fixture 341. The portions of plate screw 356 that interface with the bone fixture 341 substantially correspond to an abutment screw discussed in some additional detail below, thus permitting plate screw 356 to readily fit into an existing bone fixture used in a percutaneous bone conduction device. In an exemplary embodiment, plate screw 356 is configured so that the same tools and procedures that are used to install and/or remove an abutment screw (described below) from bone fixture 341 can be used to install and/or remove plate screw 356 from the bone fixture 341 (and thus the plate assembly 352).

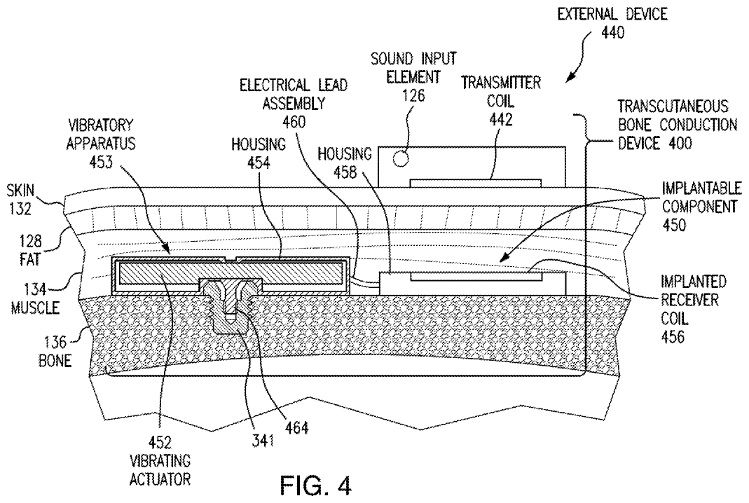

FIG. 4 depicts an exemplary embodiment of a transcutaneous bone conduction device 400 according to another embodiment that includes an external device 440 (corresponding to, for example, element 140B of FIG. 1B) and an implantable component 450 (corresponding to, for example, element 150 of FIG. 1B). The transcutaneous bone conduction device 400 of FIG. 4 is an active transcutaneous bone conduction device in that the vibrating actuator 452 is located in the implantable component 450. Specifically, a vibratory element in the form of vibrating actuator 452 is located in housing 454 of the implantable component 450. In an exemplary embodiment, much like the vibrating actuator 342 described above with respect to transcutaneous bone conduction device 300, the vibrating actuator 452 is a device that converts electrical signals into vibration.

External component 440 includes a sound input element 126 that converts sound into electrical signals. Specifically, the transcutaneous bone conduction device 400 provides these electrical signals to vibrating electromagnetic actuator 452, or to a sound processor (not shown) that processes the electrical signals, and then provides those processed signals to the implantable component 450 through the skin of the recipient via a magnetic inductance link. In this regard, a transmitter coil 442 of the external component 440 transmits these signals to implanted receiver coil 456 located in housing 458 of the implantable component 450. Components (not shown) in the housing 458, such as, for example, a signal generator or an implanted sound processor, then generate electrical signals to be delivered to vibrating actuator 452 via electrical lead assembly 460. The vibrating electromagnetic actuator 452 converts the electrical signals into vibrations.

The vibrating electromagnetic actuator 452 is mechanically coupled to the housing 454. Housing 454 and vibrating actuator 452 collectively form a vibrating element 453. The housing 454 is substantially rigidly attached to bone fixture 341.

Some exemplary bone fixtures that correspond to bone fixture 210 will now be described.

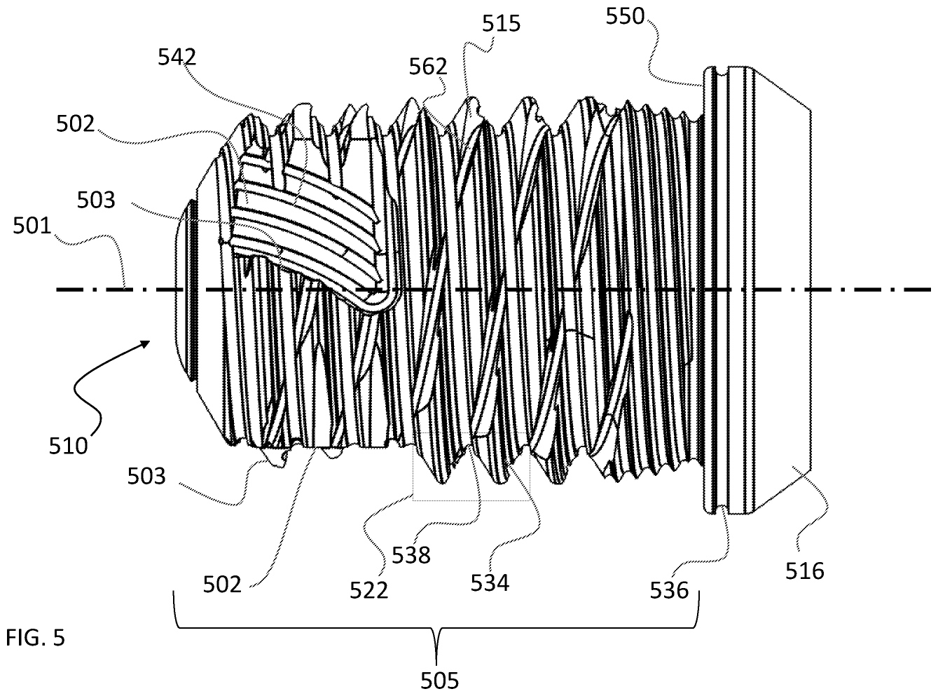

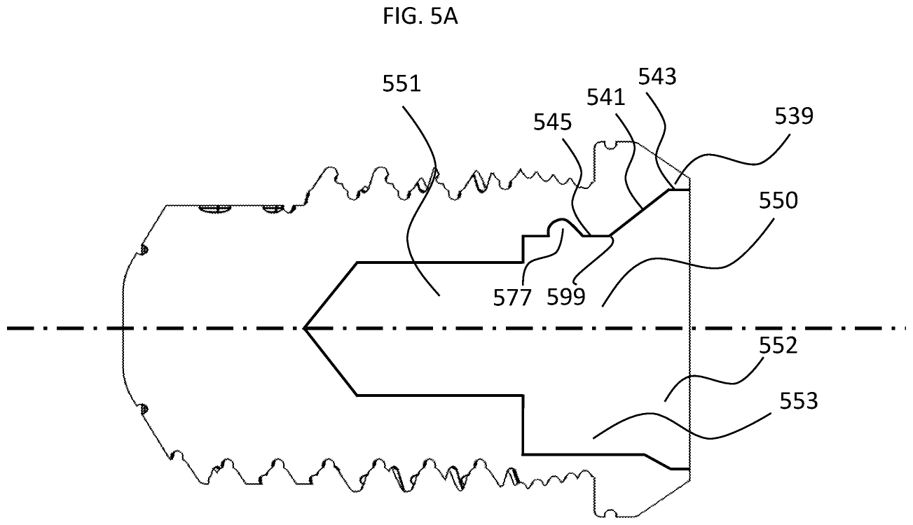

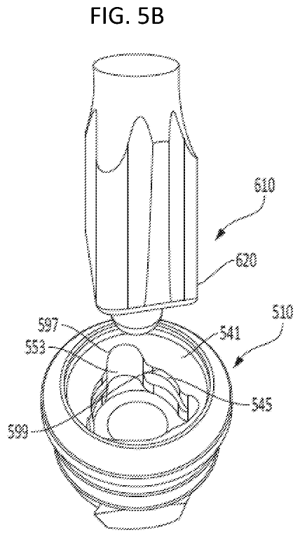

FIG. 5 depicts an exemplary embodiment of a bone fixture 510. In an exemplary embodiment, bone fixture 510 corresponds to bone fixture 210 of FIG. 2. Bone fixture 510 includes a screw thread 515 configured to screw into a skull, corresponding to thread 215 of FIG. 2. In an exemplary embodiment, the pitch of the screw thread 515 is between about 0.2 to about 1.00 mm or any value or range of values therebetween in about 0.01 mm increments (e.g., about 0.3 to about 0.8 mm). In an exemplary embodiment, the depth of the thread is between about 0.1 to about 1.25 mm or any value or range of values therebetween in about zero 0.1 mm increments (e.g., about 0.25 to about 0.8 mm).

In an exemplary embodiment, bone fixture 510 has a section 522 having such non-uniformity. As can be seen, the thread angle of the section is asymmetrical.

It is noted that in an alternate embodiment, the faces of the thread can be compound faces. That is, for example, one face of the thread may have a first surface that extends at a first angle from the centerline 523, and a second surface that extends at a second angle from the centerline 523 different from the first angle.

As can be seen in FIG. 5, the thread sections have grooves on the flanks thereof. More particularly, FIG. 5 depicts a portion 532 of the bone fixture 510 encompassing two sections of thread. As can be seen, the proximally facing flanks of the thread (i.e., the flanks of the thread that face the flange 516, or more particularly, face the bottom surface 550 of the flange 516 of the bone fixture 510, and thus face towards the proximal end of the bone fixture) have grooves 534 therein. It is noted that in alternative embodiment, the distally facing flanks of the threads (i.e., the flanks of the thread that face the end of the bone fixture 510/face away from the end that has the flange 516) have grooves therein.

It is noted that cross-section of the grooves 534 can be substantially hemispherical with the “equator” aligned/flush with the top face of the thread.

Again with reference to FIG. 5, it is noted that in some embodiments, the flange 516 of the bone fixture 510 includes a groove 536. It is noted that in the embodiments where the flange 516 includes groove(s), the grooves can correspond to any of the grooves detailed herein and/or variations thereof and/or any other shaped groove that can have utilitarian value and/or otherwise can enable the teachings detailed herein and/or variations thereof to be practiced.

Again with reference to FIG. 5, some embodiments include grooves 538 at the root (i.e., where the flanks of the thread converge, also referred to as base) of the thread. As can be seen, the root of the thread has a groove 538 therein. In an exemplary embodiment, the depth of the grooves 538 is in the range of about 50 to 200 μm, and the width of the grooves 538 is in a range of about 70 to 250 μm.

FIG. 5A presents a partial cross-sectional view of the bone fixture of FIG. 5. Here, there is a bore 550 to receive the abutment screw. The inside of the bore is threaded, at least partially (area 551 is threaded, for example). The bore opens up into a cavity 552 that is established by wall/berm 539, which wall/berm extends about the longitudinal axis of the bone fixture. Also shown is a canted wall surface 541, which extends from the wall surface 543 inwardly to wall surface 545. As can be seen, the canted wall surface 543 does not extend as far on one side as it does on the other. This is because there is a cavity 553 for a wrench to interface with so as to impart a torque to the fixture. This is seen in FIG. 5B, which depicts a top isometric view of the fixture, along with a tool 610, the details of which will be described below. Also seen in FIG. 5A is edge 599, which represents the boundaries of canted wall surface 543 and wall surface 545. As can be seen, the layout of the interior of the upper portions of the longitudinal axis interior of the bone fixture are symmetrical about planes lying on the longitudinal axis and parallel thereto which are 120° from each other. Thus, there are three (3) cavities 553 for the three lobes 620 of the tool 610. There are also three edges 599, in view of the fact that in between each of the cavities 553 there exists the greater extending canted wall 541. (Note that in this embodiment, the edges 599 flow into the corresponding edges that establish the boundaries of the canted wall 541 and the cavities 553, and is indicated as edge 597 in FIG. 5B.)

As noted above, the exemplary embodiments of the bone fixture 510 of FIG. 5, includes a flat section 502. In an exemplary embodiment, the flat section 502 is a cutting pocket extending across two or more thread crests relative to the longitudinal axis 501 of the bone fixture 510 (the embodiment depicted in FIG. 5 has a cutting pocket extending across three thread crests).

In an exemplary embodiment, the pockets 502 provide for respective cutting edge lines 503, where the edge lines 503 is defined by the edges of the thread. In an exemplary embodiment, the cutting pockets 302 in general, and the edge lines 503 particular, provide a self-tapping functionality of the bone fixture 510.

In an alternate embodiment, the cutting edge 503 can spiral in a direction consistent with the direction of the thread 515.

Some additional details of how the tool 610 operates with respect to the bone fixture will be described in greater detail below. First some additional details of the tool 610.

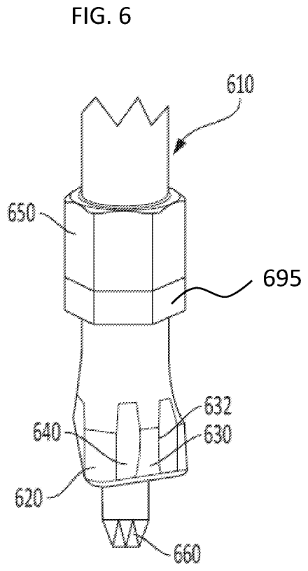

FIG. 6 presents an isometric view of a bottom portion of a tool 610. In an exemplary embodiment, the tool can be machined from a round stock of stainless steel, etc., although the tool could be machined from a bar stock or the like. Various geometries can be achieved depending on the ease or difficulty of machining the various components. Indeed, there are a myriad of ways to produce the tool as well as a myriad of ultimate configurations of the tool. In the embodiments seen in FIG. 6, the main body of the tool 610 is cylindrical, and is interrupted by wrench flats 650 and the working end of the tool, which includes the lobes 620. The opposite end of the tool, which is not shown, can include a configuration that will enable the tool 610 to be readily connected to a drill motor or a wrench motor so as to apply a torque to the tool 610. In such an embodiment, the wrench flats 650 can establish a first torque receiver section and the configuration that will enable the tool 6 tend to be readily connected to a drill motor establish a second torque receiver section (the second torque transfer section can be flats and/or can be a T-shaped or a C-shaped coupling, or could be a round body to which a drill chuck could grip). FIG. 6 also shows wrench flats 695, which can be of a different size than wrench flats 650 (size with respect to wrench) and/or can be angled relative to the flats 650, to provide different angles and/or to enable the use of different sized wrenches. Three or four or five or more wrench flat components can be used instead of one or two.

The lobes 620 represent a torque transfer section. The lobes 620 fit into the hollows 553 inside the fixture 510. The tool is used screw the fixture 510 into bone.

The tool depicted in FIG. 6 has been machined such that there are valleys 640 on either side of the lobes 620. In an exemplary embodiment, valleys 640 separate the lobes. In some embodiments, where there are three lobes, there are also three valleys. That said, in the embodiment depicted in FIG. 6, there are structures located in between each of the lobes 620. These structures represent a rise from the valleys 640. In this exemplary embodiment, the structures create two valleys 640 between the lobes 620. The structures respectively rise up from the two valleys 6402 and edge 632, which establishes a boundary between the valleys 640 and a surface 630. In the embodiment presented in FIG. 6, the surface 630 is a plane surface that is canted with respect to the longitudinal axis of the tool 610. Accordingly, in the embodiment of FIG. 6, where the working end of the tool 610 and/or the area below the wrench flats 650 is symmetrical about three planes spaced apart at 120° from each other that lie on and are parallel to the longitudinal axis of the tool 610, there are six valleys 640, respective to valley pairs separating the lobes 620, of which there are three, from each other, and there are three structures in between each of the respective two valley pairs.

The structures establish three wedge components, which wedge into the inside of the fixture 510 and retain the fixture 510 to the tool 610.

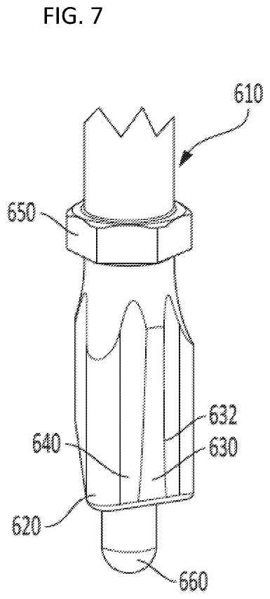

FIG. 7 presents an alternate example of the tool 610. As will be understood below, the specific arrangement of the tool can vary widely, and there are many configurations that can be utilized with the fixture 510. It is believed by the inventors that the arrangement of FIG. 6 will have commercial appeal in that it is aesthetically pleasing owing to its “all business” look.

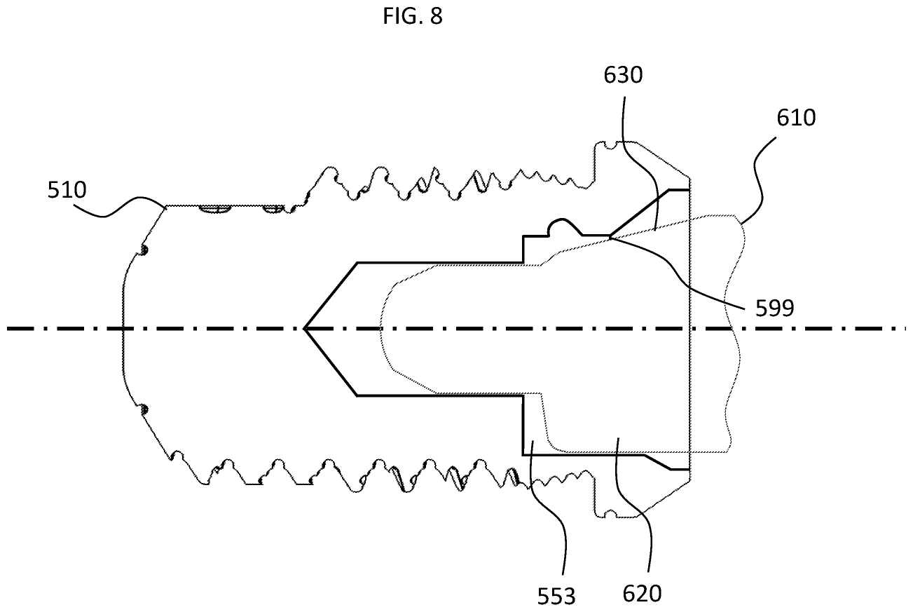

FIG. 8 presents a cross-sectional view of the fixture 510 and the tool 610 with the tool interfacing with the fixture. Here, surface 630 is seen relative to edge 599, when lobe 630 is located in the hollows 553. It is noted that surface 630 is slightly offset from the location where the edge 599 is represented in the cross-sectional view. This is because the flat surfaces 630 of the structures do not conform to the curved nature of edge 599. In this regard, each of the structures has two line contacts or two-point contacts with the edge 630 (one of the edges 632 of the surface 630 contacts the edge 599 on one side, and the other of the edges 632 of the surface 630 contacts the edge 599 on the other side—in total, there can be six contact locations).

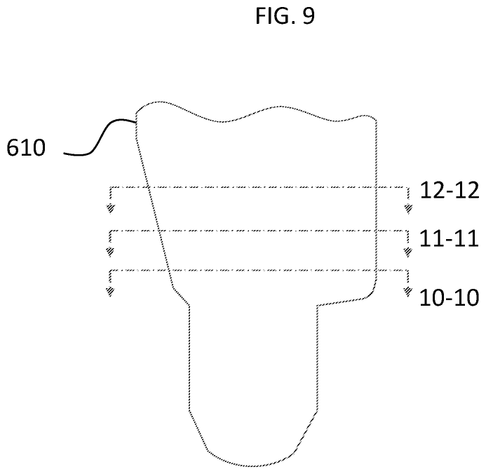

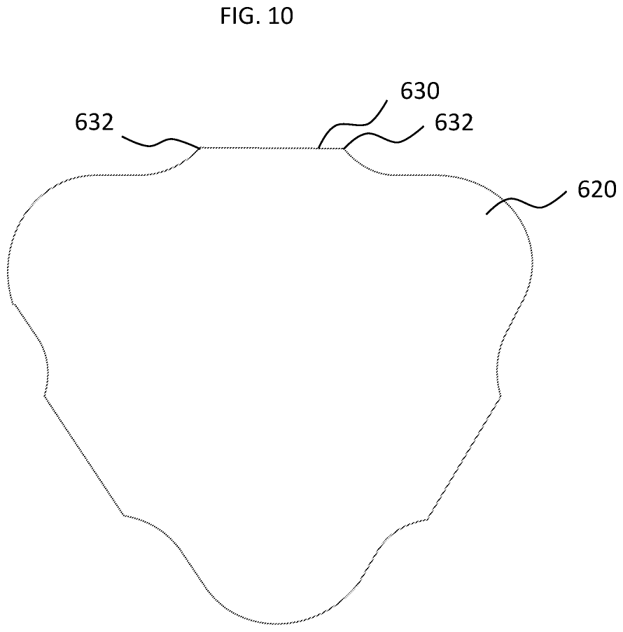

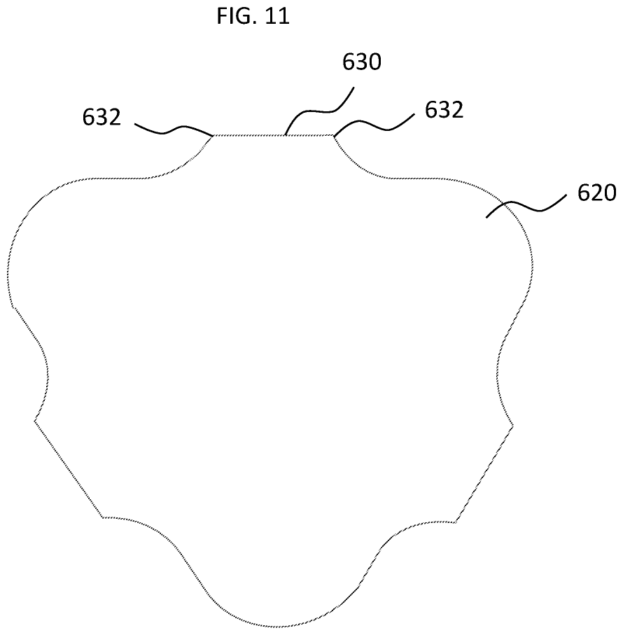

FIG. 9 presents a silhouette of a side view of a portion of the tool 610, with cross-sectional symbols presented there on. FIG. 10 represents the cross-sectional view of section 10-10 of FIG. 9, while FIG. 11 represents the cross-sectional view of section 11-11 of FIG. 9, and FIG. 12 represents the cross-sectional view of section 12-12 of FIG. 9. As can be seen, the height of the surface 630, and thus the height of the edges 632 from the longitudinal axis and/or geometric center increases progressively with location along the longitudinal axis away from the distal end. This creates the wedge shape of the three structures referenced above. In this regard, by varying the angle that the surface 630 extends away from the geometric center and/or longitudinal axis, the contact location of the edges 632 with the edge 599 can be varied. It is noted that a wide range of angles could be used that would result in a wide range of contact locations, and the specific location of contact is somewhat arbitrary and otherwise can be irrelevant.

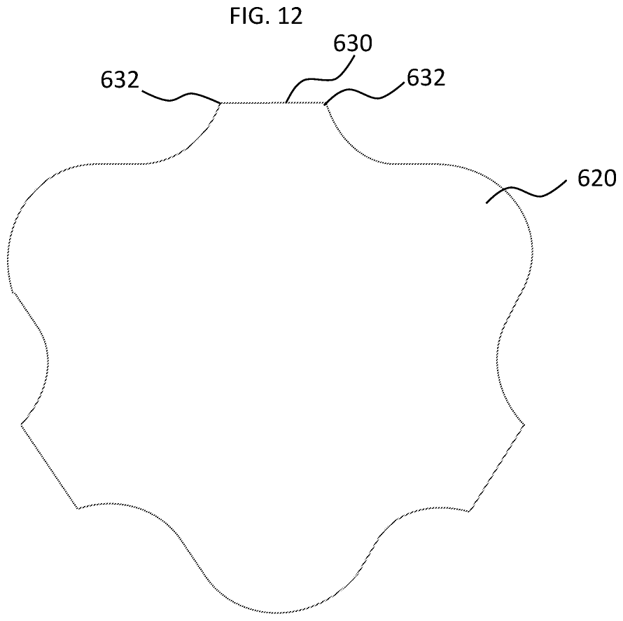

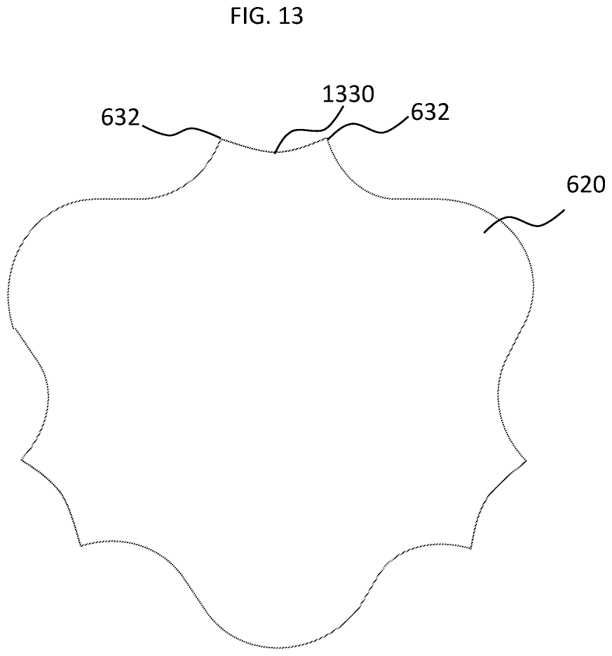

Indeed, while the embodiments above have focused on a flat surface 630, it is noted that many other types of surfaces can be utilized, such as surface 1330 of FIG. 13, which is concave relative to the outside world, or surface 1430, which is convex relative to the outside world. In both of these embodiments, edges 632 are still present, even though the flat surface 630 does not exist. Still, it has been deemed by the inventors that the flat surface provides an aesthetically appealing feature that contrasts well with the curved lobes 620. It is also noted that in at least some exemplary embodiments, the radius of curvature of surface 430 is larger than the radius of curvature of edge 599, such as by way of example, 1.1, 1.2, 1.3, 1.4, 1.5, 1.6, 1.7. 1.8, 1.9, 2, 2.25, 2.5, 2.75, 3, 3.5, 4, 4.5, 5, 5.5, 6. 7, 8, 9, 10, 11, 12, 13, 14, or 15 times or more or any value or range of values therebetween in 0.01 increments, depending on how the device is to look.

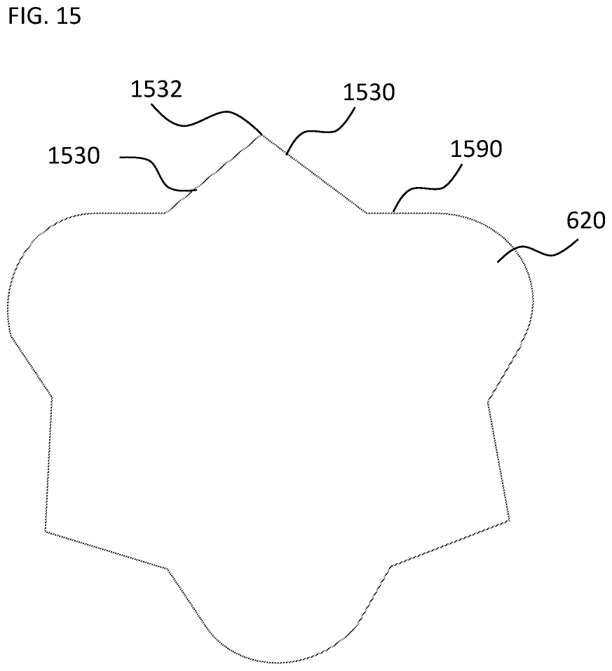

FIG. 15 presents an alternate exemplary embodiment where instead of edges 632, there is a single edge 1532 established by the junction of surfaces 1530 which extend away from surfaces 1590 which extends away from surface 1532 to lobes 620 and otherwise is blended into the lobes 620. In this exemplary embodiment, there would be three line or three point contact with the edge 599 instead of the six liner point contact of the embodiments above.

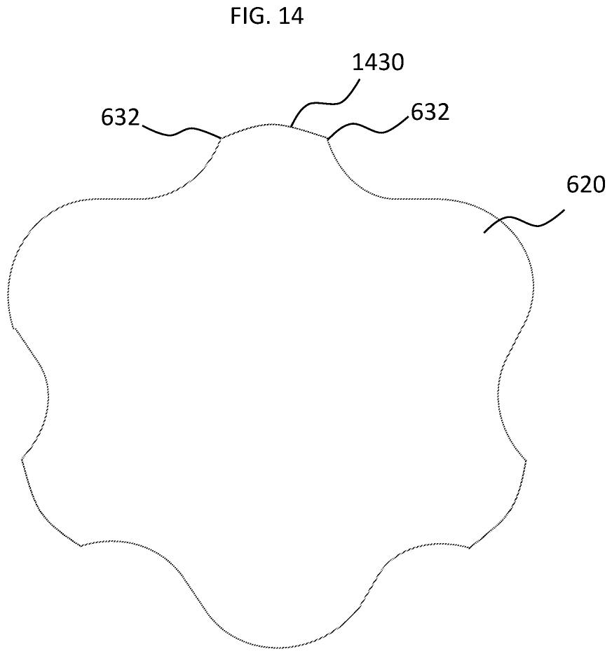

It is also noted that the various features can be combined. In some embodiments, the structure of FIG. 10 could be used on one side, the structure of FIG. 14 can be used on the other side, and the structure of FIG. 15 can be used on another side. However, this would likely be aesthetically unpleasing, and thus a uniform or symmetrical device is likely to be more appealing. As will be further detailed below, any feature of any embodiment detailed herein can be combined with any other feature with any other embodiment providing that the market would deem such to be desirable. Also, any feature of any embodiment detailed herein can be eliminated from any embodiment and/or otherwise excluded from combination with any other feature of any other embodiment.

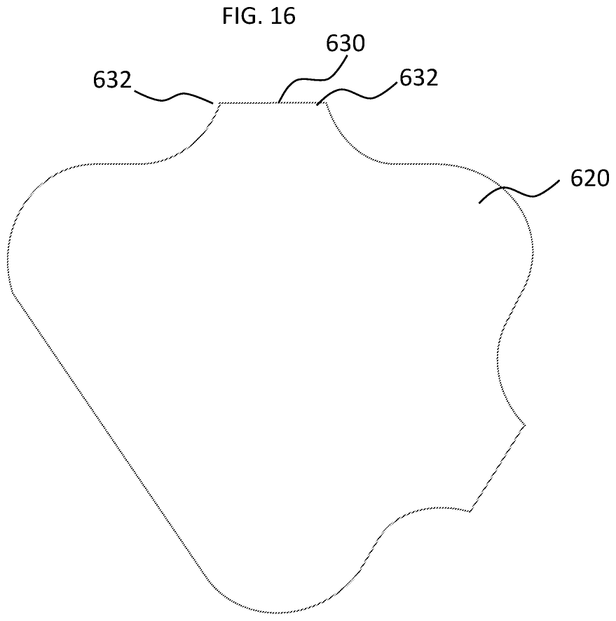

In this regard, FIG. 16 presents an exemplary embodiment where there are only two structures instead of three. While this embodiment would work as well as any of the other embodiments detailed herein, and in fact would likely be easier to make, this looks odd and otherwise would be the less aesthetically pleasing than the embodiments detailed above. Still, the point is that features can be added or excluded to achieve a desired result.

It is briefly noted that while FIGS. 13, 14, and 15 depict only single cross-sections for the respective configurations, the features would vary in a manner analogous to how the features of FIGS. 10, 11, and 12 vary with location along the longitudinal axis.

It is also noted that the location of the cross-sections of FIGS. 10, 11, and 12 are arbitrary, and other cross-sections at other locations would vary in a similar manner in at least some exemplary embodiments accordingly.

The edges 632 and/or 1532 can be utilized to create a slight interference fit and/or a friction fit with the fixture 510 at the edge 599 which will result in a modicum of retainment of the fixture to the tool 610. By retaining the fixture 510 to the tool, the fixture can be moved without touching the fixture other than the tool, and thus the tool can lift the fixture and otherwise move the fixture by moving the tool and not touching the fixture by hand.

It is also noted that a male protrusion 660 is located at the distal end in some embodiments, while in other embodiments, this is not present. From a visual standpoint, the protrusion 660 is appealing in that it would appear to guide the tool into the threaded bore of the fixture 510. In practice, however, the actual guiding will occur by trying to align the lobes with the hollows. By rough analogy, a blunt car key has become ubiquitous (to the extent that car keys are still utilized), and the user knows to align the flat sides of the car key with the rectangular hole in the ignition.

In view of the above, there is a device such as a bone fixture insertion tool, comprising a body, such as the body seen in FIG. 6 or FIG. 7. The body includes a torque transfer section, such as the lobes 620, and a torque receiver section, such as the flats 650. This device is a bone conduction hearing prosthesis bone fixture implant insertion device configured to interface with an interior of the bone fixture, as seen.

In some embodiments of this device, the device is made of a same class of materials at the torque transfer section and the torque receiver section and section(s) that interface with the bone fixture (which would include the structure in between the lobes, and the lobes, and, if present, the protrusion 660). By class of the material, it is meant steel based (steel alloy), titanium based (titanium alloy), plastic based, wood based (not that one would do that—this simply gives an example of material type. Thus, if the protrusions 620 were made out of titanium and the portion that establishes the flats 650 were made out of stainless steel, this would be different classes of material. In some embodiments, the portion between the flats 650 and the lobes can be made of titanium, and if the portion that establishes the flats 650 and the lobes 620 and the structure that establishes surface 630 were made out of stainless steel, this would meet the aforementioned requirement about the same class of materials at the torque transfer section and the torque receiver section and the sections that interface with the bone fixture.

In an exemplary embodiment, the torque transfer section and the torque receiver section and section(s) that interface with the bone fixture are integral sections of the body (and, in some embodiments, the entire tool). In an exemplary embodiment, the section(s) that interface with the bone fixture include the torque transfer section and a bone fixture interference section (which can be the structure that establishes the edges 632, which interfere with the bone fixture 510, some additional details of which will be provided below). Corollary to this is that in some embodiments, the section(s) that interface with the bone fixture can include the torque transfer section and a bone fixture interference section that includes three separate interference zones (e.g., respective zones that respectively include the respective groups of edges 632 that contact with the bone fixture during use. In some embodiments, the section(s) that interface with the bone fixture include the torque transfer section and a bone fixture interference section that includes six separate interference surface portions (e.g., the six edges 632) that contact with the bone fixture during use.

As noted above, in some embodiments, the device is configured to lift and retain the bone fixture to the device during use. In an exemplary embodiment, the tool 610 can be pushed into the bone fixture 510 with sufficient force and/or with sufficient velocity such that the edges 632 gouge or otherwise plastically deform edge 599. In an exemplary embodiment, six V shaped depressions would be made in the edge 599 with a vector approximately normal to the respective edges 632 of the tool 610. In an exemplary embodiment, this plastic deformation would result in an interference fit and/or a friction fit at those locations. In this exemplary embodiment, the plastic deformation would result in adherence of the bone fixture to the tool. That said, in at least some exemplary embodiments, the deformation of the bone fixture is instead elastic deformation. That is, in some exemplary embodiments, there is no plastic deformation.

In some exemplary embodiments, the minimum amount of force that is required to withdraw the fixture from the tool is X, where X can be 0.1, 0.2, 0.3, 0.4, 0.5, 06, 0.7, 0.8, 0.9, 1.0, 1.25, 1.5, 1.75, 2, 2.25, 2.5, 2.75, 3, 3.25, 3.5, 3.75, 4, 4.5, 5, 5.5, 6, 6.5, 7, 7.5 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100 Newtons or more or any value or range of values therebetween in 0.01 Newton increments (e.g., 30.3N, 15.5N, 6.66N to 37.77N, etc.). In an exemplary embodiment, the minimum amount of force that is required to attach the fixture to the tool is also X (but it would likely be different than the minimum amount of force that is required to withdraw the fixture from the tool—X is used as a variable to illustrate possible scenarios). In an exemplary embodiment, the aforementioned values can be the maximum amount of force that is required to withdraw the fixture from the tool and/or to attach the fixture to the tool.

It is noted that in at least some exemplary embodiments, the lobes 620 are sized and dimensioned to establish a slip fit or a clearance fit with the portion of the housing that establishes the hollows for the lobes. In this regard, in an exemplary embodiment the lobes have nothing to do with retaining the bone fixture to the tool. It is further noted that in an exemplary embodiment, the protrusion 660, if present, also can have nothing to do with retaining the bone fixture the tool. In other embodiments, this is not the case, the protrusion and/or the lobes can play a part in retaining the fixture to the tool.

The aforementioned values are values with respect to forces applied in the direction of the longitudinal axes of the tool and the bone fixture. It is noted that there can be other regimes of releasing the attachment of the fixture to the tool, such as applying a torque in a direction in a plane that is parallel to the longitudinal axis while holding the bone fixture so that it cannot move or otherwise resist the torque. Some additional details of this will be described below.

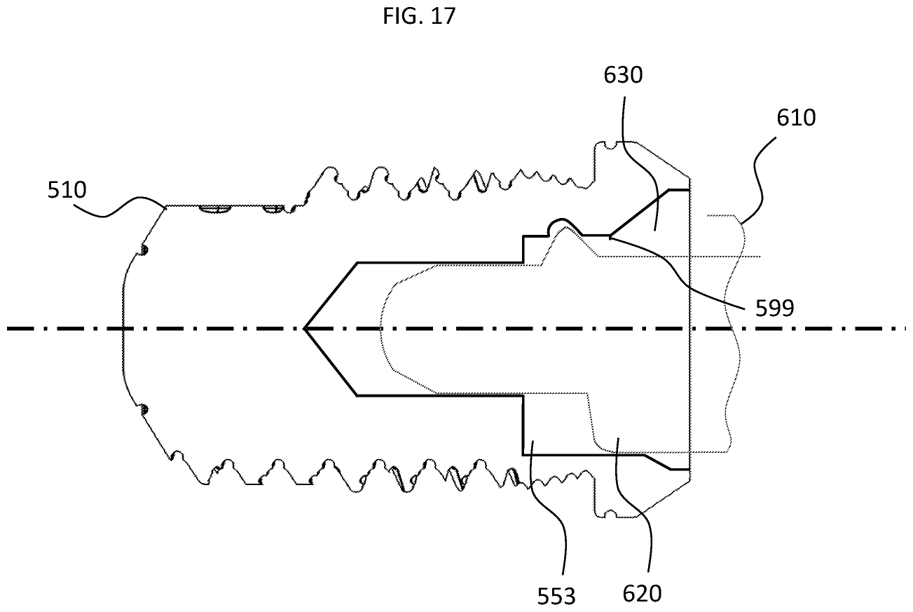

In any event, it can be seen that in at least some exemplary embodiments, the device is configured to lift and retain the bone fixture to the device during use without positive interference with the bone fixture (such as would be the case if, for example, the tool reached into groove 577, as seen in FIG. 17, which is not encompassed in any means for gripping of this application).

In an exemplary embodiment, the section(s) that interface with the bone fixture include the torque transfer section and a bone fixture interference section that includes at least one flat surface obliquely angled relative to a longitudinal direction of the body, which flat surface creates line interference contact with the bone fixture during use, which lines of the line interference established boundaries of the flat surface. That said, as noted above, in some embodiments, the section(s) that interface with the bone fixture include the torque transfer section and a bone fixture interference section that includes at least one curved surface (concave or convex relative to the outside world obliquely angled relative to a longitudinal direction of the body, which flat surface creates line interference contact with the bone fixture during use, which lines of the line interference established boundaries of the flat surface.

In an exemplary embodiment, the body is a monolithic component (e.g., machined from a single piece of stainless steel). In an exemplary embodiment the entire device is a monolithic component. That is, there is

With respect to the longitudinal axis of the tool, from a location of interface of the tool with the bone fixture to a distal end of the tool which fits into the component, a cross-sectional areal lying normal to the longitudinal axis reduces or remains constant with location. Indeed, as seen above, this can be the case with respect to a location that is a few millimeters above the location of interface. In an exemplary embodiment, this phenomenon exists with respect to a distance of the tool from the distal end, but starting at that distance working towards the distal end, of Y, where Y can be 1, 1.25, 1.5, 1.75, 2, 2.25, 2.5, 2.75, 3, 3.25, 3.5, 3.75, 4, 4.25, 4.5, 4.75, 5, 5.25. 5.5. 5.75, 6, 6.25, 6.5. 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25. 15.5. 15.75, 16, 16.25, 16.5. 16.75, 17, 17.25, 17.5, 17.75, 18, 18.25, 18.5, 18.75, 19, 19.25, 19.5, 19.75, 20, 20.25, 20.5, 20.75, 21, 21.25, 21.5, 21.75, 22, 22.25, 22.5, 22.75, 23, 23.25, 23.5, 23.75, 24, 24.25, 24.5, 24.75, 25, 25.25. 25.5. 25.75, 26, or any value or range of values therebetween in 0.01 mm increments.

FIG. 18 depicts a detailed view of an abutment 2 connected to a bone fixture 1 by an abutment screw 3. The bone fixture 1 can correspond to the bone fixture 510 above or any other bone fixture. In this embodiment, the bone fixture 1 has a main body that has a tapered apical proximate end 1A. The distal region 1B of fixture 1 is fitted with three self-tapping cutting edges formed into the exterior surface of the fixture.

There is a flange 7 that has a planar bottom surface 10 for resting against the outer bone surface, when anchoring fixture 1 has been screwed down into the skull bone.

The outer peripheral surface of flange 7 has a cylindrical part 12 and a flared top portion 13. A tapered inner side wall 14 is adjacent to the grip section (not shown). The interior of the flange 7 further includes an inner bottom bore 15 having internal screw threads for securing a coupling shaft 3 (abutment screw).

The skin penetrating part of the implant includes the abutment sleeve 2. In one embodiment, abutment sleeve 2 has an inner annular flange 16 at its upper edge 17A configured to cooperate with a removable component of a bone conduction device, consistent with that seen in FIG. 2, via a snap-in connection or the like. Abutment sleeve 2 has an internal shoulder with a central opening for a connecting shaft or coupling shaft 3 and a number of peripherally arranged through holes or recesses 19 used for a tool that will be detailed below.

The three components (abutment 2, bone fixture 1 and abutment screw 3) establish the implantable portion of a percutaneous bone conduction device. In this regard, this can be considered the implantable assembly. As will be detailed below, in some embodiments, the entire assembly is implanted into a recipient at one time as an assembly, while in other embodiments, after the bone fixture 1 (or 510) is implanted into the recipient, such as by utilizing tool 610, the abutment 2 is then attached to the bone fixture via abutment screw 3 (as opposed to already having been attached). Tools that are utilized in both scenarios will now be described.

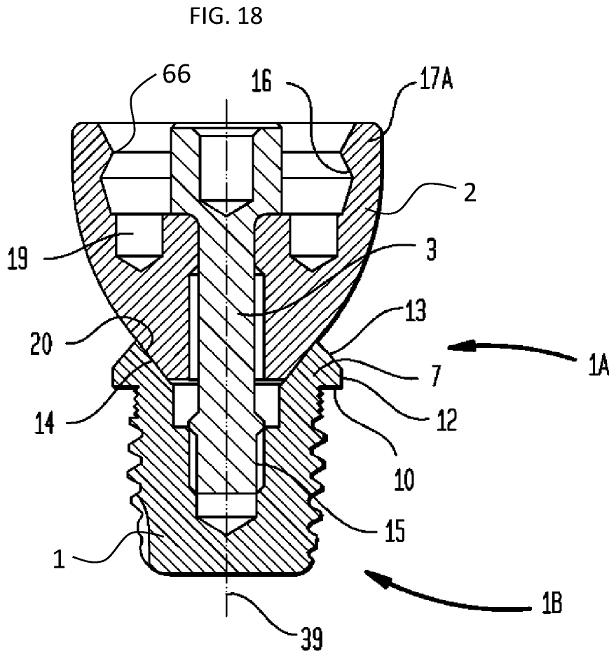

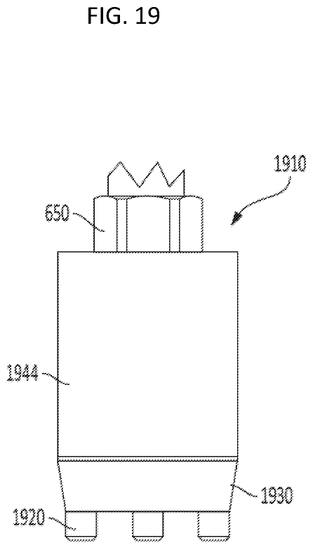

FIGS. 19 and 20 depict a tool 1910 that is configured to lift and install an implant assembly, such as the implant assembly shown in FIG. 18, into a human. In an exemplary embodiment, the tool 1910 includes a torque receiver section established by flats 650 and a torque transmission section established by rounds/protrusions 1920. Surface 1930 establishes an abutment interface surface that is configured to interface with a portion of the abutment and also provide a friction and/or an interference fit so that the abutment and the rest of the assembly can be lifted and moved with the tool 1910 in a manner analogous to the tool 610 detailed above for the bone fixture. Here, however, the tool 1910 is lifting the assembly that includes the bone fixture and the abutment. That said, in an exemplary embodiment, the tool 1910 can be configured to lift only the abutment, or, more accurately, the tool can be used to lift on the abutment.

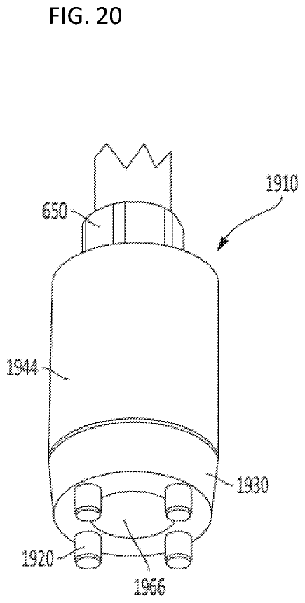

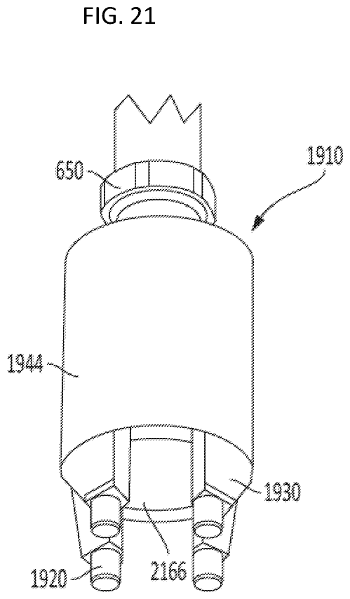

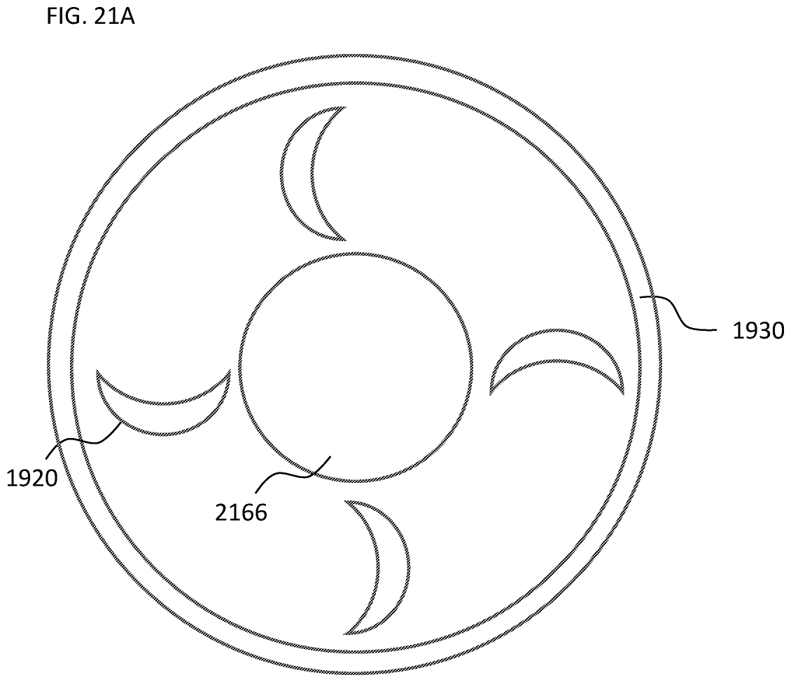

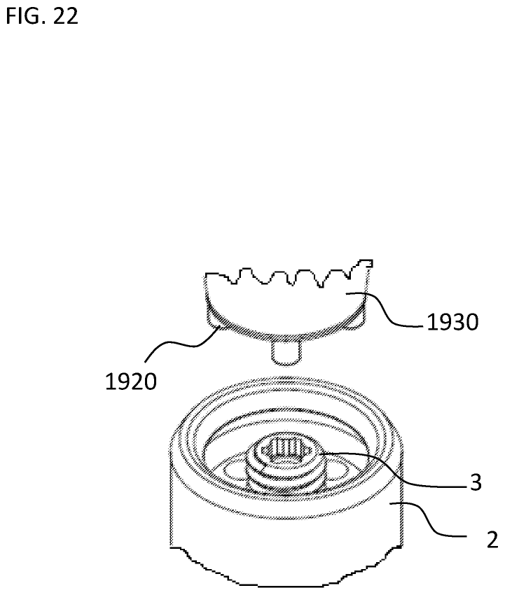

Briefly, there is a hole 1966 that provides a clearance for the abutment screw 3 when present. The configuration shown in circular, but any shape hole can be used that will enable clearance. Indeed, it is not necessary that this device be manufactured as seen in the figures. FIG. 21 presents an alternate embodiment where there is no hole per se, but instead simply prongs and surface portions where there are passageways 2166. Moreover, square or rectangular or half-moon or crescent moon protrusions can be used instead of the round protrusions. Indeed, a crescent moon configuration arrayed in a pattern could give the appearance of rotation and thus be visually appealing (e.g., such as that seen in FIG. 22, which depicts a bottom view of a tool that looks really cool). Also, while the bottoms of the protrusions are depicted as flat, in an alternative embodiment, the bottoms of the protrusions can be concave or convex relative to the outside of the tool (curved when viewed from the side, or conical surfaces). Indeed, in this regard, while probably meaningless, a concave shape might give the appearance of a less-intrusive device (as opposed to a convex surface, which would give the appearance of a stabbing or thrusting device—where the use of the tool would not want to cut/hurt the skin during use).

FIG. 22 presents an isometric view of the bottom portion of the tool 1910 approaching the top portion of the abutment 2 and the abutment screw 3, where this abutment 2 is a bit different than the abutment shown above in that the top of the abutment is cylindrical for more of a distance along a longitudinal axis than the embodiment shown above.

FIG. 23 depicts an exemplary scenario where the tool is inserted into the abutment 2 in a manner that the abutment 2 is retained to the tool. Only the abutment 2 is shown. It is to be understood that in an alternative embodiment, the abutment 2 is connected to the bone fixture 1 by abutment screw 3, with the space in between the two protrusions 1920 providing clearance for the top of the abutment screw 3. In this configuration, surface 1930 is in contact with edge 66, which establishes the innermost portion of the female portion of the snap coupling of the bone conduction hearing prostheses of FIGS. 2A and 2B (edge 66 is a boundary of the recess 221 with respect to the configuration of FIGS. 2A and 2B, for example, and forms the innermost portion of overhang 222, and is the innermost portion of the sidewall of FIG. 2B). In this regard, edge 66 is the demarcation point between the inward profile of the interior of the abutment that “compresses” the male portions of the snap coupling of the removable component of the bone conduction device and the outboard profile of the interior of the abutment that permits the mail portions of the snap coupling of the removable component to snap outward and thus be secured underneath the edge 66/the lip that is established by that structure.

FIG. 24 depicts an alternate embodiment of the tool, or at least the working end of the tool, where the protrusions 1920 have different end shapes and have different lengths than that of FIG. 23. As can be seen, this is aesthetically unpleasing and illustrates the aesthetically pleasing nature of the configuration of FIG. 23.

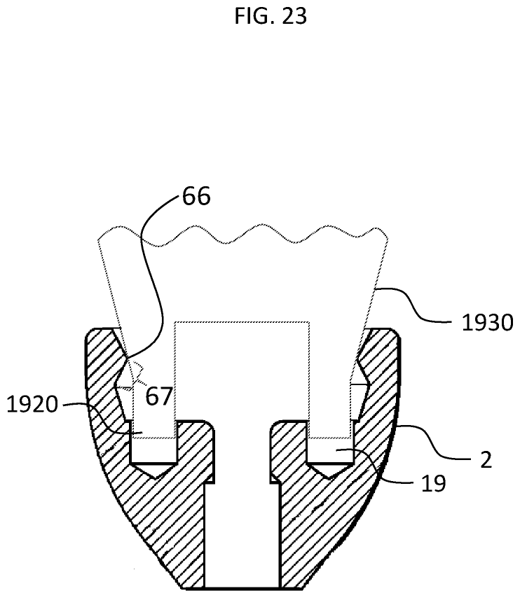

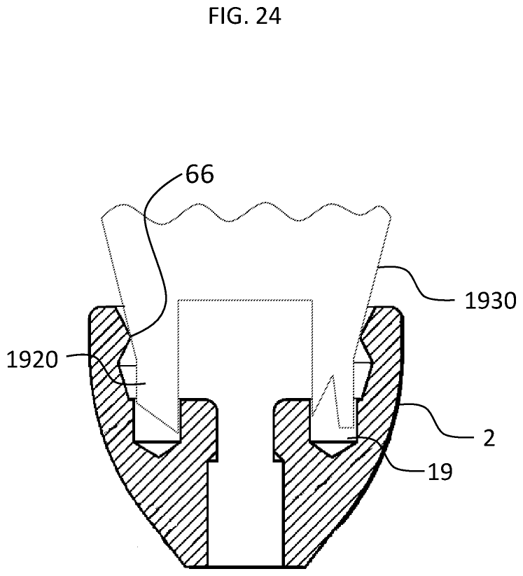

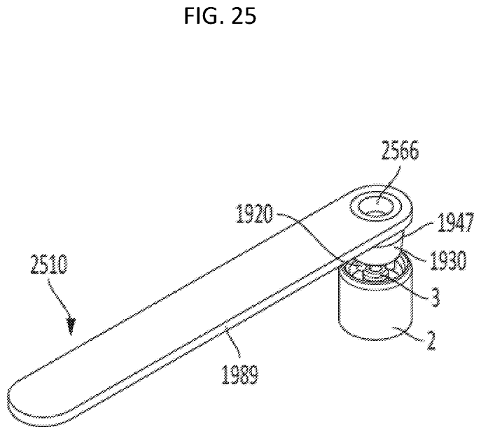

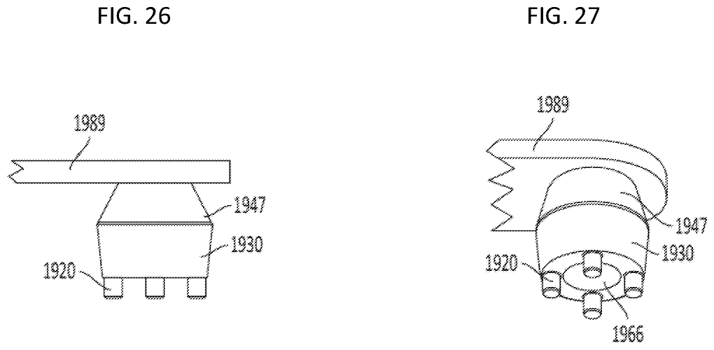

FIG. 25 depicts an alternate tool, 2510, that uses some of the features of tool 1910. However, tool 2510 is a tool that is configured to apply a counter torque to the abutment 2 as opposed to applying a torque to the abutment 2. In this regard, while the embodiment of tool 1910 is configured to apply a torque to the assembly of the abutment and the bone fixture when held together with the abutment screw, the embodiment of tool 2510 is configured to be used when the abutment is not secured to the bone fixture/is configured to be used while the abutment screw 3 is being torqued to secure the abutment 2 to the bone fixture 1. In an exemplary embodiment, tool 2510 can also be used to torque the assembly of the abutment and bone fixture into and/or out of the bone. (It is noted that all tools herein can be used for removal. Accordingly, any disclosure of implantation corresponds to an alternate embodiment where there is instead removal/the method actions may be executed in reverse as detailed herein.) FIGS. 26 and 27 depict partial views from different perspectives of the tool of FIG. 25.

(It is briefly noted that all references to abutment 2, bone fixture 1, and abutment screw 3 correspond to a reference in an alternate embodiment to the other bone fixtures and abutments and abutment screw as detailed herein and/or variations thereof and/or other such functional devices in the art.)

Still with reference to FIG. 25, tool 2510 includes a handle 1989 which has a hole 2566 therethrough at one end to which is attached a structure 1947. Structure 1947 parallels at least some of the features of tool 1910 detailed above, at least with respect to the working end of the tool. Indeed, in an exemplary embodiment, structure 1947 constitutes a cutoff of an existing tool 1910 combined with attachment to a handle 1989 (e.g., by welding or by an interference fit, shrink fit, etc.). In an exemplary embodiment, through hole 1966 extends all the way through the structure to hole 2566. This provides access for a tool, such as an Allen wrench, to reach the abutment screw 3. More specifically, in an exemplary embodiment, after the bone fixture has been implanted in bone of a recipient, tool 2510 is utilized to transport or otherwise move abutment 2 to a location proximate/on the bone fixture 1, and then prevent the abutment 2 from rotating while torque is applied to the abutment screw 3 to tighten and secure the abutment 2 to the bone fixture 1. It is briefly noted that while not shown in the embodiments of the figures, in an exemplary embodiment, the abutment 2 has a distal end that includes lobes analogous to the lobes of the tool 610 above, that fits into the corresponding hollows of the bone fixture. Accordingly, in an exemplary embodiment, if some torque that is applied to the abutment screw 3 causes the abutments and/or the bone fixture to rotate, the tool 2510 prevents the abutment and thus the bone fixture from rotating in reaction to that torque, at least when the prongs/protrusions 1920 are located in the holes 19 on top of the abutment 2. In this regard, in an exemplary embodiment, a surgeon or other healthcare professional holds the handle 2510 while the torque is being applied to the abutment screw 3 utilizing an Allen wrench or the like extending through hole 2566 and hole 1966, and thus reacts any torque that exists so as to not interfere with the placements/securement of the already implanted bone fixture in the bone.

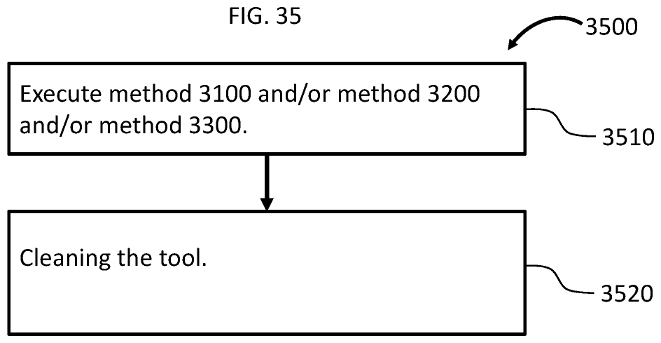

In an exemplary embodiment, to the extent that the tool is made of two or more parts and/or that the body (which can be a sub-portion of the tool, where the body can be monolithic but there is a component attached to the body that makes the overall tool not monolithic—in an exemplary embodiment, the component shown/body shown in the figures are monolithic in some embodiments—in an exemplary embodiment, the component/body that extends from and includes the wrench flats to the distal end are monolithic) the parts are not loosely connected to each other (e.g., there is no ring that can move in its entirety relative to another component of the tool). For example, the protrusions at the bottom of tool 1910 can be interference fitted into the structure that establishes the conical surface 1930, but if the interference fit is sufficiently tight/strong enough, the protrusions (which could be dowels) will not be loose relative to the remainder of the tool. FIGS. 35 and 36 depict an alternate embodiment of a tool, where structure 1947 is not canted as in the above. In some embodiments, structure 1947 can be canted outward (the opposite of that of FIGS. 26 and 27) with respect to location from bottom to top. Any arrangement can be utilized providing that it does not interfere with usage of the tool.

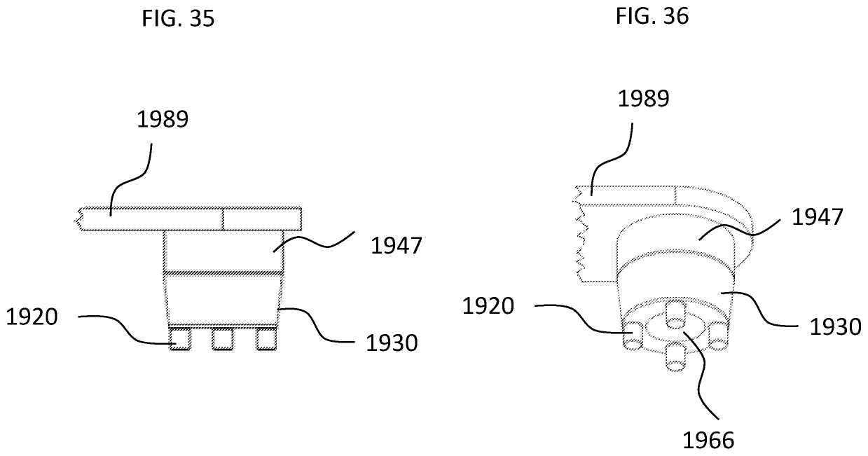

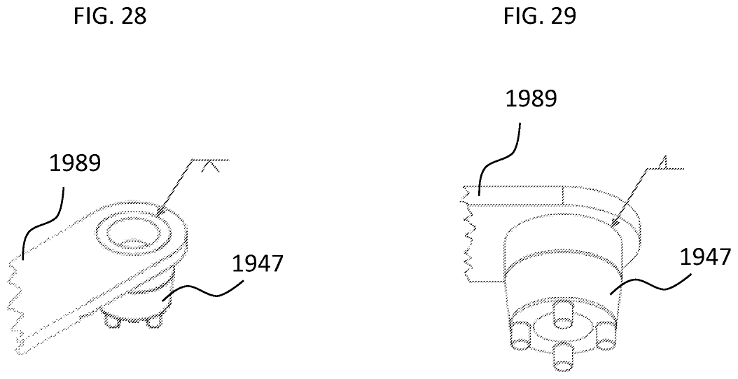

Of course, in an exemplary embodiment, structure 1947 can be made explicitly and purposely for the tool 2510. Indeed, in an exemplary embodiment, as with tool 610 and tool 1910, tool 2510 can be made of a monolithic component, while in other embodiments, structure 1947 is interference fitted or slip fitted to the handle 1989 (in an exemplary embodiment, a larger hole bored through the handle 1989, and the handle 1989, which is made of stainless steel, for example, is heated to a temperature of maybe 300 or 400 degrees F., thus expanding the hole, and then the structure 1947 is placed into the hole, and as the handle cools, the hole shrinks around the structure 1947 thus attaching the structure 1947 to the handle 1989, thereby establishing a shrink fit. Alternatively, and/or in addition to this, a weld can be applied at an interface between the two tools—top and/or bottom, as seen in FIGS. 28 and 29 (weld symbols approximate). This can have utilitarian value with respect to eliminating any gap or crevice that might retain dirt or bacteria or the like (at least after the weld is ground down flush with the structure 1947 and handle 1989—a notch can be put into the material and then filled with welled, and a fillet could be left over on the bottom side, which fillet would be smooth after the grinding—the welds might not be that seen in FIGS. 28 and 29, to achieve this—any weld that can enable such can be used in some embodiments). This would not be monolithic, but would be an integral component.

In an exemplary embodiment, the body that establishes the working portions of the tool is monolithic, and that body is coated with TiN, for example, at least in part.

In view of the above, in an exemplary embodiment, there is a device, comprising a torque transfer section (the section including the protrusions 1920, or the handle 1989), and a torque receiver section (the section including the flats 650, or the protrusions 1920, in the case of the counter torque tool 2510), wherein the device is a bone conduction hearing prosthesis abutment implantation device (which includes tool 1911 and tool 2510, as both are used to implant the abutment, albeit the former as part of the overall assembly of the implant) configured to interface with an interior of the abutment. In an exemplary embodiment, the device is made of like materials at the torque transfer section and the torque receiver section and section(s) that interface with the bone fixture (surface 1930 and protrusions 1920, for example). By like materials, this can be titanium and stainless steel, as these do not negatively react with one another (as opposed to graphite and titanium, for example). In this regard, in an exemplary embodiment, the protrusions 1920 can be titanium dowels that are inserted into holes bored into the body 1947 (in the case of a non-monolithic body). In an exemplary embodiment, dowels are welded to the body and then the weld is smoothed as necessary to result in the above-noted elimination of crevices, to the extent that a tight interference fit would not result in such. Note that a friction weld might be usable.

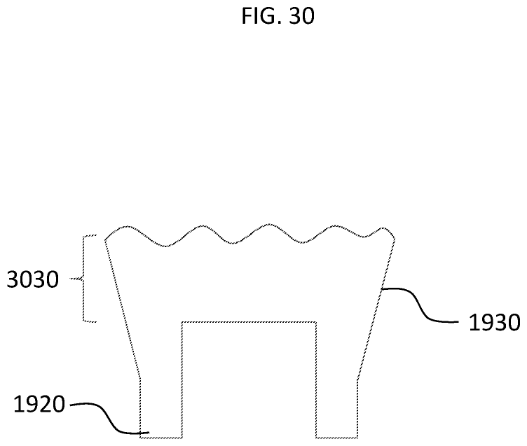

In an exemplary embodiment, the device is configured to retain the abutment to the device and the device is configured such that torque transferred from the device is applied through a component that retains the device to the abutment. In this regard, portion 3030 (see FIG. 30) of body 1947 that has the surface 1930 transfers all torque to and/or from the protrusions 1920. This is because the torque that is applied at the flats and/or the protrusions 1920 must travel through the portion 3032 reach the protrusions 1920 or reach the handle. Accordingly, this feature is present in both tool 1910 and tool 2510.

It is noted that the conical surface 1930 has some relationship to the features associated with the structure establishing the edges 632. In this regard, in at least some exemplary embodiments, any of the features detailed above with respect to the structure establishing the edges 632 and/or the surface 630 and/or the variations thereof (edge 1532, the inverted surfaces, etc.). Accordingly, at least some exemplary embodiments include any disclosure detailed above with respect to the tool 610 is applicable to the tool 1910 and/or the tool 2510, unless otherwise stated providing that the art enables such, and vis-a-versa. By way of example only and not by way of limitation, any disclosure herein associated with the edges of the structure (e.g., the angle away from the longitudinal axis, the distance of extension along the longitudinal axis, etc.) is applicable to the surface 1930, and vice versa. Of course, with respect to interface features, the interface would be with respect to the abutment instead of the bone fixture.

Briefly, for example, the device is made of a same class of materials at the torque transfer section and the torque receiver section and section(s) that interface with the abutment (instead of the bone fixture). The torque transfer section and the torque receiver section and section(s) that interface with the abutment are integral sections of a body establishing such and/or the entire tool. In an exemplary embodiment, the section(s) that interface with the abutment include the torque transfer section and an abutment interference section. In an exemplary embodiment, the section(s) that interface with the abutment include the torque transfer section and an abutment interference section that includes 1, 2, 3, 4, 5, 6, 7, 8, 9, or 10 or more separate interference zones that contact with the abutment during use (this can be the case for the tool for the fixture, where it would be bone fixture interference section).

In an exemplary embodiment, the section(s) that interface with the abutment include the torque transfer section and an abutment interference section that includes 1, 2, 3, 4, 5, 6, 7, 8, 9, or 10 or more separate interference surface portions that contact with the abutment during use (this can be the case for the tool for the fixture, where it would be bone fixture interference section). As with tool 610, except with a different component, the device is configured to lift and retain the abutment to the device during use. The device is configured to lift and retain the abutment to the device during use without positive interference with the abutment (e.g., such as gripping/extending underneath edge 66—analogous to how what is seen in FIG. 17 operates, gripping/extending underneath an outer lip that extends on an outside of an alternate embodiment of an abutment, extending sufficiently far around the arc of the outside of the abutment such that that is positive retention, etc.). In an exemplary embodiment, the section(s) that interface with the abutment include the torque transfer section and a bone fixture interference section that includes at least one obliquely angled surface relative to a longitudinal direction of the body/tool, which surface creates interference contact with the bone fixture during use. In an exemplary embodiment, the device is a monolithic component. In an exemplary embodiment, the tool has a longitudinal axis and from a location of interface of the tool with the component to a distal end of the tool which fits into the component, a cross-sectional areal lying normal to the longitudinal axis reduces or remains constant with location.

In an exemplary embodiment, the device is configured such that when interfacing with the abutment, with respect to respective outermost profiles of the device and the abutment (as opposed to, an inner profile, such as hole 1966), the interface is completely male-female relationship with the device being the male part. This as opposed to a configuration, for example, that has structure that envelops an outer portion of the abutment. In an embodiment, the device is configured such that when interfacing with the abutment, the device provides a retaining feature that retains the abutment to the device at a location that is susceptible to wear due to removal and attachment of a bone conduction removable component to the abutment. In this regard, the inner annular flange 16/the edge 66/the overhang 222/the surface established by the recess formed by sidewall 221 are areas of the abutment that are susceptible to wear owing to the repeated removal and attachment of the external component of the bone conduction device to the abutment, where these surfaces elastically deform the teeth of the snap coupling to retain the coupling to the abutment and release coupling from the abutment. Over time, this can create wear on the abutment. Also, to the extent that there is an area that is critical or otherwise important in the interior of the abutment (at least one that is not associated with a bacterial route from the outside to the inside, such as around the abutment screw and down through the bore into the recipient), this is one of the most critical areas, because if the geometry of this area changes over time, the external component will not be as rigidly retained or otherwise as utilitarianly retained, which can result in a decrease in the magnitude of an outputted vibration, and/or an attenuation of certain frequencies under all frequencies (it could also permit the external component to rattle relative to the abutment which would cause unwanted sound or otherwise would be irritating). The point is, the area at issue of the abutment is an area that should be handled as carefully as possible. In this regard, the art would eschew utilizing this area as a handling point, but the tools detailed herein do so.

In an exemplary embodiment, the surface(s) of the abutment that interface with the tool/the tool interfaces with surface(s) of the abutment, such as interior surface of the abutment that experience alternating contact and lack of contact with the external component/removable component of the bone conduction device, which alternating contact/lack of contact can be 30, 40, 50, 60, 70, 80, 90, 100, 150, 200, 250, 300, 350, 400, 500, 600, 700, 800, 900, 1000, 1250, 1500, 1750, 2000, 2500, 3000 or more or any values or range of values therebetween in one increment cycles (on/off), wherein the surfaces of the abutment at issue are configured to withstand such cycling as a normal design characteristic. This distinguishes form, for example, an outer surface of the abutment, or the surface underneath the abutment screw (which would see some wear/cycling if the abutment screw is untightened and then retightened), but nowhere near the cycling of removal and attachment of the removable device.

Consistent with tool 610, the device is a combined lifting and torque adapter for the abutment (as opposed to tool 610, which is for the fixture, of course), and, for embodiments with a clearance for the abutment screw, a combined lifting and torque adapter for the abutment-fixture assembly (which would be included in the “adapter for the abutment”). In an exemplary embodiment, the device is a combined lifting and counter torque wrench for the abutment.

The device can be configured such that when interfacing with the abutment, the device provides a retaining feature that retains the abutment to the device solely by friction. In some embodiments, all interfacing surfaces with the abutment are part of a monolithic portion of the body. In some embodiments, the torque transfer section has a cross-section lying on a plane that is normal to a longitudinal axis which includes six protrusions outward away from the longitudinal axis, which protrusions are distinct from each other and form an overall profile of the tool.

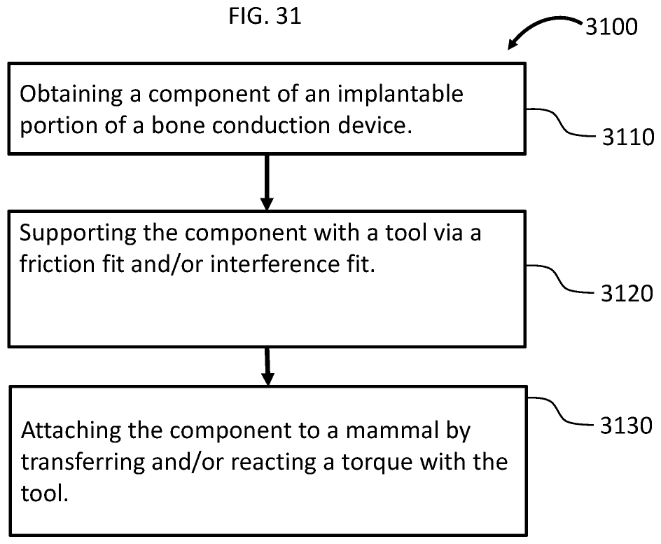

An exemplary embodiment, actually, a few of them, includes methods. In this regard, by way of example only, FIG. 31 presents an exemplary flowchart for an exemplary method, method 3100. Method 3100 includes method action 3110, which includes the action of obtaining a component of an implantable portion of a bone conduction device. This could be a bone fixture, an abutment, and an assembly of a bone fixture and an abutment (held together by an abutment screw, for example). Note also that in at least some exemplary embodiments, a single piece/monolithic combined bone fixture and abutment may be the component of the implantable portion of a bone conduction device, which configuration was somewhat more prevalent in the 2000's and the 20^{th} Century. Accordingly, any disclosure herein of handling an abutment and for implanting an abutment corresponds to a disclosure of handling an assembly of an abutment combined with a fixture in a single piece abutment-fixture combination. Also note that the component can be a fixture of a passive transcutaneous bone conduction device or an active transcutaneous bone conduction device, such as the devices of FIGS. 3 and 4 above.

In an exemplary embodiment, method 3100 further includes method action 3120, which includes the action of supporting the component with a tool via a friction fit and/or interference fit. This can be done with any of tools 610, 1910 and 2510 as detailed above and/or variations thereof. In an exemplary embodiment, method action 3120, in fact, the entire method 3100 for that matter, is executed without utilizing a positive retention vis-à-vis a tool that is utilized to support the component and the abutment and/or fixture. Method 3100 further includes method action 3130, which includes the action of attaching the component to a mammal by transferring and/or reacting a torque with the tool. Again, this can be done with any of the tools 610, 1910, in combination with a drill motor, or a wrench, or some other torque producing/torque transferring device, vis-à-vis transferring torque, or with tool 2510, in combination with an Allen wrench or the like, vis-à-vis reacting a torque with the tool.

In an exemplary embodiment, the action of supporting the component plastically deforms the component. For example, such as where tool 610 is utilized, the edges 632 push into edge 599 of the bone fixture, wedging therein, which can form a V shaped indentation at the six line/point contacts. In an exemplary embodiment, the component is a bone fixture of the bone conduction device. Conversely, in some embodiments of the tools 1910 and/or 2510, the tools do not plastically deform the component of a bone conduction device, such as the abutment, but instead, at most, elastically deform the component. In an exemplary embodiment, the tool is elastically deformed instead and/or in addition to any deformation that occurs with respect to the component of the bone conduction device, and this can be the case with respect to tool 610, tool 1910 and/or tool 2510. Accordingly, in at least an exemplary embodiment, method 3100 includes permanently deforming and otherwise damaging the bone fixture at least a little bit using the tools detailed herein and/or the methods of implantation detailed herein.

In an exemplary embodiment, the action of supporting the component (and it is noted that by “supporting,” supporting includes retaining the component relative to one or a direction that counteracts the effects of gravity, and thus does not necessarily require the force to be located at the bottom (a table supports a plate, but an electromagnet attached to a crane also supports the car body at a junkyard)) utilizes relatively sharp edges and cuts into the component, again, were, for example, the component is a bone fixture of the bone conduction device.

Still further, in an exemplary embodiment of method 3100, the action of supporting the component is executed by establishing a friction fit at a first surface of the component. This can be executed utilizing tool 1910 and/or tool 2510, and in some embodiments, tool 610, such as where, for example, the structure establishes a curved surface having a radius of curvature (e.g., lying on a plane normal to the longitudinal axis) that is about the same as the radius of curvature of the edge 599. In an embodiment where the component is an abutment of a percutaneous bone conduction device, the first surface is a surface that holds a removable component of the bone conduction device to the abutment (e.g., the surfaces detailed above, for example).

Consistent with the teachings above, in some embodiments of method 3100, the tool is not positively retained to the component during the transferring of or reaction to the torque.

In some embodiments of method 3100, the component is an abutment of a percutaneous bone conduction device, and the tool is metal at all locations that interface with the abutment. That is, there is no component of the tool that is not metal that contacts the abutment. In an exemplary embodiment of method 3100, the component is an abutment or a bone fixture of a bone conduction device, and there is no portion of the tool that contacts the abutment and/or bone fixture that is made of a material of a different class and/or a different type than another portion of the tool that contacts the abutment and/or all portions of the tool that contact the abutment and/or fixture are of like material and/or are the same material (e.g., all 316 stainless steel).

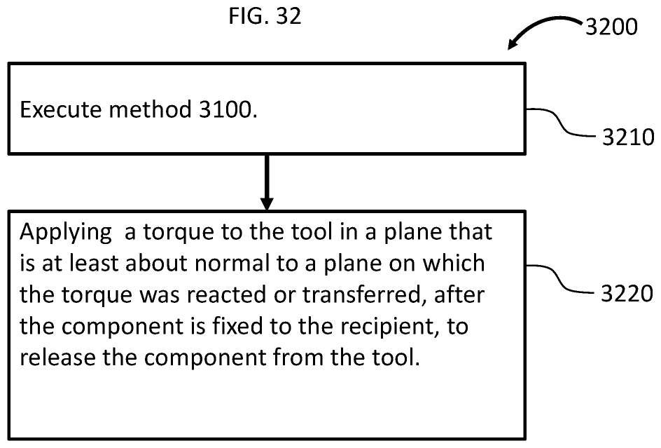

FIG. 32 presents an exemplary flowchart for another exemplary method, method 3200, which method includes method action 3210, which includes executing method 3100. Method 3200 further includes method action 3220, which includes the action of applying a torque to the tool in a plane that is at least about normal to a plane on which the torque was reacted or transferred, after the component is fixed to the recipient, to release the component from the tool. (It is briefly noted that any of the method actions detailed herein can be executed in any order providing that the art enable such. Accordingly, the methods detailed herein can be met by practicing the method actions irrespective of the order, unless otherwise noted.) This regard, in at least some exemplary embodiments, canting the tool relative to the longitudinal axis can “break” the retention forces that are present that hold the given component of the tool. By way of example only and not by way of limitation, a force applied at a distance away from the fixture to the tool 610 in the plane of FIG. 8 or normal to the plane of FIG. 8 could aid in freeing the tool 610 from the fixture 510/make it easier to remove. This combined with a slight upward force away from the fixture could result in ease of detachment relative to that which would be the case if the tool was pulled directly away from the fixture. This is also the case with respect to the abutment.

In an exemplary embodiment, a torque applied according to method action 3220 results in a reduction of directly away force that is required to separate the tool from the component by 5, 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98 or 99% or any value or range of values therebetween in 0.1% increments relative to that which would be the case without that force applied according to method action 3220.

It is noted that in some exemplary embodiments, the local radius of curvature/a radius of curvature lying on a plane normal to the longitudinal axis of surface 1930 can correspond to that of edge 66, and can be within plus or minus 0.1, 0.2, 0.3, 0.4, 0.5, 0.6. 0.7, 0.8, 0.9, 1, 1.25, 1.5, 1.75, 2, 2.25, 2.5, 2.75, 3, 3.5, 4, 4.5, 5, 5.5, 6, 6.5, 7, 7.5, 8, 8.5, 9, 10, 11, 12, 13, 14, 15, 16, 17, 18, 19, or 20% or more or any value or range of values therebetween in 0.01% increments (where the radius of curvature of the edge is the denominator, and the radius of curvature of the tool is the numerator. This can also be the case for the bone fixture tool vis-à-vis the interfacing edge as well. This could create a situation where there is no plastic deformation of the abutment and/or bone fixture.

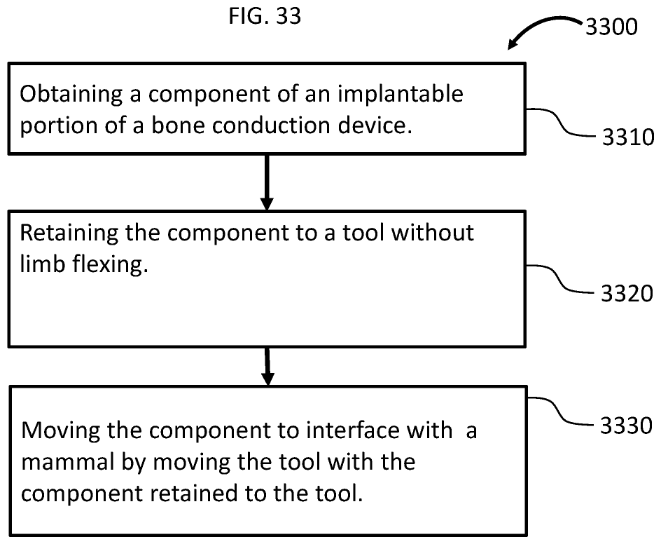

FIG. 33 presents an exemplary flowchart for an exemplary method, method 3300, which includes method action 3310, which includes obtaining a component of an implantable portion of the bone conduction device. This could be a bone fixture, and abutment, the bone fixture abutment assembly, the bone fixture and abutment unitary implant, etc. Method 3300 further includes method action 3320, which includes retaining the component to a tool without limb flexing. In this regard, prior art tools have limbs, such as a flexible ring that fits into the area 577, and such as teeth. These are limbs in that they are a component that extends away from another component. Indeed, the protrusions are 1920 are limbs, but there is no flexure in the tool during retaining action. The structure that establishes the edges 632 might be considered a limb, and any plastic deformation that occurs is not flexure (by analogy, a body builder may “flex” his or her muscles, but a twitch is not flexing one's muscles even though the muscles move, by analogy, a bumper of a car does not flex while driving down the road, even though an outer profile may vary a bit, but it does flex when striking another bumper at a low speed that does not leave any permanent damage (save for some surface scuffing).

Method 3300 further includes method action 3330, which includes moving the component to interface with a mammal, such as a human, by moving the tool with the component retained to the tool. It is noted that this is the case with respect to the anti-torque wrench/counter torque wrench where such is used to only move the abutment to the bone fixture already implanted into the recipient as that falls within the scope of interfacing with a mammal, even though no tissue may be directly contacting the abutment at that specific time (skin will be pulled away, for example, and the abutment will be separated from direct contact by bone by the bone fixture).

In an exemplary embodiment, there is a method that expands method 3300, where the method further includes the action of applying a torque to the component via a first portion of the tool. In this exemplary embodiment, the torque is reacted on the component at a location that has a locational component that is at a same level, along a longitudinal axis of the component, as a location where the component was retained to the tool (e.g., this results from tool 610 being used with a bone fixture). In some embodiments, the component is an abutment of a percutaneous bone conduction device, and an outer surface of the abutment at a topmost portion is generally cylindrical. This, as contrasted, with an abutment that has a lip, for example, that establishes the outermost profile of the abutment. Corollary to this is in at least some exemplary embodiments, the component is an abutment that does not have the aforementioned lip. In an exemplary embodiment, the abutment has an outer surface that is an outermost profile that has a constant distance from the longitudinal axis of the abutment completely about the outside of the abutment, which constant distance is the case for locations along the longitudinal axis over a distance of less, than, equal to or greater than 0.1, 0.2, 0.3, 0.4, 0.5, 0.6. 0.7, 0.8, 0.9, 1, 1.25, 1.5, 1.75, 2, 2.25, 2.5, 2.75, 3, 3.5, 4, 4.5, 5, 5.5, or 6 mm, or any value or range of values therebetween in 0.01 mm increments. The cylindrical nature of the outside of the abutment could extend over at least some of these distances.

That said, in some embodiments, again, where the component is an abutment of a percutaneous bone conduction device, an outer surface of the abutment at a topmost portion has a lip extending outward. In this regard, at least some exemplary embodiments of the tool detailed herein can operate with an abutment that has the outer lip and one that does not have an outer lip. Indeed, in some exemplary embodiments, the tool detailed herein can be utilized with an abutment that has the aforementioned constant distance from the longitudinal axis for less than 0.1 mm. Still further, embodiments of the teachings detailed herein can be utilized with abutments that have an outer surface that has a varying distance from the longitudinal axis over the first 0.1, 0.2, 0.3, 0.4, 0.5, 0.6. 0.7, 0.8, 0.9, 1, 1.25, 1.5, 1.75, 2, 2.25, 2.5, 2.75, 3, 3.5, 4, 4.5, 5, 5.5, or 6 mm from the top of the abutment. Thus, the tools detailed herein can operate over a wide variety of abutment shapes.

Consistent with the teachings above, in an exemplary variation of method 3300, the method further includes affirmatively canting the tool relative to the component, at least after the component is fixed to the recipient, to release the component from the tool. This as opposed to directly pulling the tool away from the component. Indeed, in an exemplary embodiment, the methods are executed without pulling the tool directly away from the component and/or by imparting a force directly away from the component that is less than the force that is utilized to cant the tool, and the force maybe less than the force that is utilized to cant the tool by any of the aforementioned amounts.

Again, variations in the methods detailed herein further include the application of a torque to the component. In at least some exemplary embodiments, the force(s) resulting in the retention of the component of the tool are at least partially overcome during the action of applying torque to the component. By way of example only and not by way of limitation, once the torque is applied to the abutment, that very well may break any retention. Conversely, in an exemplary embodiment, the application of torque can increase the retention force and/or increase the amount of force that is required to remove the component from the tool. In an exemplary embodiment, as a result of the application of torque, the amount of force that is required to remove the component from the tool can be decreased, and in other embodiments increased, by less than, more than or equal to 5, 10, 15, 20, 25, 30, 35, 40, 45, 50, 55, 60, 65, 70, 75, 80, 85, 90, 95, 100, 125, 150, 175, 200, 225, 250, 275, 300, 350, or 400 percent or more, or any value or range of values therebetween in 1% increments relative to that which would be the case in the absence of the torque.

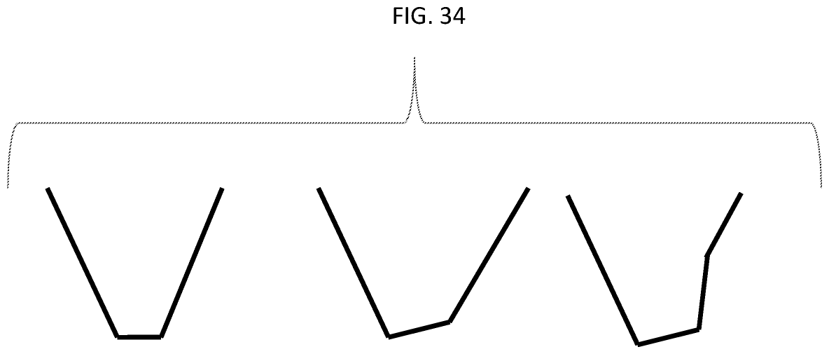

Embodiments include applying a torque to the component using the tool to screw the component into bone of the mammal, wherein the mammal is a human, wherein the action of applying the torque scores the component and/or the action of retaining the component to the tool permanently deforms the component. In an exemplary embodiment, the action of applying the torque results in the edges 632 making (leaving), instead of a V shape, a shape like one or more of those shown in FIG. 34.

In some embodiments, during the action of moving the component, no part of the tool is located underneath a part of the component. That said, a tray or a surgeon's hand might be located underneath.

It is noted that in at least some exemplary embodiments, the surface 630/edges thereof and surface 1930 has an angle that is less than, greater than or equal to about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 degrees or any value or range of values therebetween in 0.1 degree increments, and this angle can extend over a distance in an unvarying manner of less than, greater than or equal to 0.3, 0.4, 0.5, 0.6. 0.7, 0.8, 0.9, 1, 1.25, 1.5, 1.75, 2, 2.25, 2.5, 2.75, 3, 3.5, 4, 4.5, 5, 5.5 or 6 mm with respect to location along the longitudinal axis and/or with respect to distance of the surface or edge (which would be a fraction of the distance along the longitudinal axis, because of the sine or cosine effect).

In an exemplary embodiment, one or more surfaces of the tool are coated in TiN, such as the flat or comparable surfaces of tool 632, or the cone surface of tool 1910, or the entire tool or the body portion that forms the working surfaces, etc.

FIG. 35 presents an exemplary flowchart for an exemplary method, method 3500. Method 3500 includes method action 3510, which includes executing method 3100 and or method 3200 and/or method 3300. Method action 3510 can include executing any one or more the method actions detailed herein singularly and/or in combination. Method 3500 further includes method action 3520, which includes cleaning the tool after executing the method and or method action detailed herein. In an exemplary embodiment, the tool(s) are cleaned in a manner meeting Regulation (EU) 2017/745 of the European Parliament and of the Council of 5 Apr. 2017 on medical devices, amending Directive 2001/83/EC, Regulation (EC) No 178/2002 and Regulation (EC) No 1223/2009, as of the requirement's written text on Sep. 2, 2019. In an exemplary embodiment, the tool is cleaned according to that directive in less than 0.25, 0.5, 0.75, 1, 1.25, 1.5, 1.75, 2, 2.5, 3, 4, 5, 6, 7, 8, 9, 10, 11, 12, 13, 14, 15, 20, 25, 30, 35, 40, 45, 50, 55, 60, 70, 80, 90, 120, 150 or 180 minutes or any value or range of values therebetween in 1 second intervals. In an exemplary embodiments, any one or more of the methods herein and/or the method actions herein are executed repeatedly using the same physical tools, with cleaning in between uses, which use/clean cycles can be to meet the method at least 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 90 or 100 or any value or range of values therebetween in one increment. For example, in an exemplary embodiment, method 3100 and/or method 3200 and/or method 3300 is execute for any of the aforementioned number of times, combined with cleaning in between each execution of each method. In an exemplary embodiment, the various methods are executed at least any of the aforementioned times, and the aforementioned requirements for cleaning, temporally and/or by the standard, are met for at least 50, 55, 60, 65, 70, 75, 80, 85, 86, 87, 88, 89, 90, 91, 92, 93, 94, 95, 96, 97, 98, 99 or 100% of the time.

It is again reiterated that in at least some embodiments, any one or more of the teachings detailed herein can be combined with any other one or more teachings detailed herein. Conversely, any one or more of the teachings detailed herein can be explicitly excluded from use with any one or more of the other teachings detailed herein. Thus, some embodiments include embodiments that specifically do not have one or more of the teachings and/or features detailed herein.

Any disclosure herein of a device and/or a system corresponds to a disclosure of a method of making that device and/or system. Conversely, any disclosure herein of a method action of making the device and/or system corresponds to a resulting device and/or system made by that method action. Any disclosure herein of a method action corresponds to a disclosure of a device and/or system for executing that method action. Any disclosure herein of a device and/or system corresponds to a disclosure of utilizing that device and/or system. Any disclosure herein of a functionality of any apparatus corresponds to a method action of executing that functionality.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

1. A device, comprising:
a body including:
a torque transfer section; and
a torque receiver section, wherein
the device is a bone conduction hearing prosthesis bone fixture implant insertion device configured to interface with an interior of the bone fixture, and
the device is made of a same class of materials at the torque transfer section and the torque receiver section and section(s) that interface with the bone fixture.

2. The device of claim 1, wherein:
the torque transfer section and the torque receiver section and section(s) that interface with the bone fixture are integral sections of the body.

3. The device of claim 1, wherein:
the section(s) that interface with the bone fixture include the torque transfer section and a bone fixture interference section.

4. The device of claim 1, wherein:
the section(s) that interface with the bone fixture include the torque transfer section and a bone fixture interference section that includes three separate interference zones that contact with the bone fixture during use.

5. The device of claim 1, wherein:
the section(s) that interface with the bone fixture include the torque transfer section and a bone fixture interference section that includes six separate interference surface portions that contact with the bone fixture during use.

6. The device of claim 1, wherein:
the device is configured to lift and retain the bone fixture to the device during use.

7. (canceled)

8. The device of claim 1, wherein:
the section(s) that interface with the bone fixture include the torque transfer section and a bone fixture interference section that includes at least one flat surface obliquely angled relative to a longitudinal direction of the body, which flat surface creates line interference contact with the bone fixture during use, which lines of the line interference established boundaries of the flat surface.

9-10. (canceled)

11. A device, comprising:
a torque transfer section; and
a torque receiver section, wherein
the device is a bone conduction hearing prosthesis abutment implantation device configured to interface with an interior of the abutment, and at least one of:
(i) the device is made of like materials at the torque transfer section and the torque receiver section and section(s) that interface with the bone fixture; or
(ii) the device is configured to retain the abutment to the device and the device is configured such that torque transferred from the device is applied through a component that retains the device to the abutment.

12. The device of claim 11, wherein:
the torque transfer section and the torque receiver section and section(s) that interface with the bone fixture are integral sections.

13. The device of claim 11, wherein:
the device is configured such that when interfacing with the abutment, with respect to respective outermost profiles of the device and the abutment, the interface is completely male-female relationship with the device being the male part.

14. (canceled)

15. The device of claim 11, wherein:
the device is a combined lifting and torque adapter for the abutment.

16. The device of claim 11, wherein:
the device is a combined lifting and counter torque wrench for the abutment.

17-18. (canceled)

19. The device of claim 11, wherein:
the device is configured to retain the abutment to the device; and
the device is configured such that torque transferred from the device is applied through a component that retains the device to the abutment.

20. The device of claim 11, wherein:
the tool has a longitudinal axis; and
from a location of interface of the tool with the component to a distal end of the tool which fits into the component, a cross-sectional areal lying normal to the longitudinal axis reduces or remains constant with location.

21. A method, comprising:
obtaining a component of an implantable portion of a bone conduction device;
supporting the component with a tool via a friction fit and/or interference fit; and
attaching the component to a mammal by transferring and/or reacting a torque with the tool.

22. The method of claim 21, wherein:
the action of supporting the component plastically deforms the component; and
the component is a bone fixture of the bone conduction device.

23. (canceled)

24. The method of claim 21, wherein:
the action of supporting the component is executed by establishing a friction fit at a first surface of the component;
the component is an abutment of a percutaneous bone conduction device; and
the first surface is a surface that holds a removable component of the bone conduction device to the abutment.

25. The method of claim 21, wherein:
the tool is not positively retained to the component during the transferring of or reaction to the torque.

26. The method of claim 21, wherein:
the component is an abutment of a percutaneous bone conduction device; and
the tool is metal at all locations that interface with the abutment.

27. The method of claim 21, further comprising:
cleaning the tool in a manner that meets European Regulation (EU) 2017/745 of the European Parliament and of the Council of 5 Apr. 2017 on medical device.

28-44. (canceled)

45. The method of claim 21, further comprising:
applying a torque to the tool in a plane that is at least about normal to a plane on which the torque was reacted or transferred, after the component is fixed to the recipient, to release the component from the tool.

46. The method of claim 21, wherein:
there is no portion of the tool that contacts the component that is made of a material of a different class than another portion of the tool that contacts the abutment and/or all portions of the tool that contact the abutment and/or fixture are of like material.

47. A method, comprising:
obtaining a component of an implantable portion of a bone conduction device;
retaining the component to a tool without limb flexing; and
moving the component to interface with a mammal by moving the tool with the component retained to the tool.

48. The method of claim 47, further comprising:
applying a torque to the component via a first portion of the tool, wherein
the torque is reacted on the component at a location that has a locational component that is at a same level, along a longitudinal axis of the component, as a location where the component was retained to the tool.

49. The method of claim 47, wherein:
the component is an abutment of a percutaneous bone conduction device; and
an outer surface of the abutment at a topmost portion is generally cylindrical.

50. The method of claim 47, wherein:
the component is an abutment of a percutaneous bone conduction device; and
an outer surface of the abutment at a topmost portion has a lip extending outward.

51. The method of claim 47, further comprising:
affirmatively canting the tool relative to the component, after the component is fixed to the recipient, to release the component from the tool.

52. The method of claim 47, further comprising:
applying a torque to the component, wherein
force(s) resulting in the retention of the component of the tool are overcome during the action of applying torque to the component.

53. The method of claim 47, further comprising:
applying a torque to the component using the tool to screw the component into bone of the mammal, wherein the mammal is a human, wherein
the action of applying the torque scores the component and/or the action of retaining the component to the tool permanently deforms the component.

54. The method of claim 47, wherein:
during the action of moving the component, no part of the tool is located underneath a part of the component.
